(19) United States
(12) Patent Application Publication
(10) Pub. No.: US 2025/0339357 A1
(43) Pub. Date: Nov. 6, 2025

(54) PROCESS FOR DYEING THE HAIR COMPRISING THE APPLICATION OF A COMPOSITION A COMPRISING TWO ALKOXYSILANES, AND THE APPLICATION OF A COMPOSITION B COMPRISING A FILM-FORMING POLYMER

(71) Applicant: L'Oreal, Paris (FR)

(72) Inventors: Alexis Liard, ST OUEN (FR); Arnaud Bonnamy, ST OUEN (FR)

(73) Assignee: L'Oreal, Paris (FR)

(21) Appl. No.: 18/993,428

(22) PCT Filed: Jul. 10, 2023
(86) PCT No.: PCT/EP2023/069074

§ 371 Date: Jan. 10, 2025

(30) Foreign Application Priority Data
Jul. 11, 2022 (FR) 2207085

(51) Int. Cl. A61K 8/58; A61Q 5/06; A61Q 5/10

(52) U.S. Cl. CPC A61K 8/585; A61Q 5/065; A61Q 5/10; A61K 2800/43; A61K 2800/5422; A61K 2800/87

(57) ABSTRACT

A subject of the present invention is a process for dyeing keratin fibres such as the hair, comprising the application to the keratin fibres of at least one composition A comprising at least one alkoxy silane chosen from the compounds of formula (I) or of formula (F) and at least one alkoxysilane of formula (II), and the application to the keratin fibres of at least one composition B comprising at least one film-forming polymer. Said composition A and/or composition B comprise at least one colouring agent chosen from pigments, direct dyes and mixtures thereof. Said compositions A and/or B are packaged in one or more spray devices.

### TECHNICAL FIELD OF THE INVENTION

A subject of the present invention is a process for dyeing keratin fibres such as the hair, comprising the application to the keratin fibres of at least one composition A comprising at least one alkoxysilane chosen from the compounds of formula (I) or of formula (I′) and at least one alkoxysilane of formula (II), and the application to the keratin fibres of at least one composition B comprising at least one film-forming polymer. Said compositions A and/or B comprise at least one colouring agent chosen from pigments, direct dyes and mixtures thereof. Said compositions A and/or B are packaged in one or more spray devices.

### CONTEXT OF THE INVENTION

In the field of dyeing keratin fibres, in particular human keratin fibres, it is already known practice to dye keratin fibres via various techniques using direct dyes or pigments for non-permanent dyeing, or dye precursors for permanent dyeing.

There are essentially three types of process for dyeing the hair:
a) “permanent” dyeing, the function of which is to afford a substantial modification to the natural colour and which uses oxidation dyes which penetrate into the hair fibre and forms the dye via an oxidative condensation process;
b) non-permanent, semi-permanent or direct dyeing, which does not use the oxidative condensation process and withstands four or five shampoo washes; it consists in dyeing keratin fibres with dye compositions containing direct dyes;
c) temporary dyeing, which gives rise to a modification of the natural colour of the hair that remains from one shampoo wash to the next, and which serves to enhance or correct a shade that has already been obtained. It may also be likened to a “makeup” process.

It is known practice to use pigments for this last type of dyeing. Specifically, the use of pigment at the surface of keratin fibres generally makes it possible to obtain visible colourings on dark hair, since the surface pigment masks the natural colour of the fibre.

However, the colourings obtained via this dyeing method exhibit the disadvantage of having poor resistance to shampooing operations and also to external agents, such as sebum, perspiration, brushing and/or rubbing actions.

Furthermore, during the application of the pigment-based dyeing composition, the deposition of colour on the surface of the keratin fibres may be non-homogeneous, i.e. the coloured coating does not cover the fibres uniformly, which may result in a lower visibility of the colour.

There is thus still a need for a process for dyeing keratin fibres, notably the hair, which has the advantage of obtaining a visible coloured coating on the hair offering good coverage of the original hair colour, while forming a coating that is persistent with respect to shampoo washing and to the various attacking factors to which the hair may be subjected such as washing and/or brushing and/or rubbing actions, without degradation of the hair.

Thus, the aim of the present invention is to develop a process for dyeing keratin fibres, in particular the hair, which has the advantage of obtaining a visible coloured coating on the hair offering good coverage of the initial hair colour, while forming a coating that is persistent with respect to shampoo washing and to the various attacking factors to which the hair may be subjected such as washing and/or brushing and/or rubbing actions, without degradation of the hair.

The applicant has discovered, surprisingly, that all of these objectives can be achieved by means of the process according to the present invention.

### SUMMARY OF THE INVENTION

A subject of the present invention is a process for dyeing keratin fibres such as the hair, comprising the following steps:
a) applying to the keratin fibres at least one composition A comprising:
at least one alkoxysilane chosen from the compounds of formula (I) or of formula (I′) as described below, oligomers thereof and/or mixtures thereof; and
at least one alkoxysilane of formula (II) as described below, oligomers thereof and/or mixtures thereof; and
b) applying to the keratin fibres at least one composition B comprising at least one film-forming polymer,
composition A and/or composition B comprising at least one colouring agent chosen from pigments, direct dyes and mixtures thereof;
composition A and/or composition B being packaged in one or more spray devices.

The present invention also relates to a kit for dyeing keratin fibres such as the hair, comprising several compartments containing:
in a first compartment, a composition A comprising:
at least one alkoxysilane chosen from the compounds of formula (I) or of formula (I′) as described below, oligomers thereof and/or mixtures thereof; and
at least one alkoxysilane of formula (II) as described below, oligomers thereof and/or mixtures thereof;
in a second compartment, a composition B comprising at least one film-forming polymer, composition A and/or composition B comprising at least one colouring agent chosen from pigments, direct dyes and mixtures thereof;
composition A and/or composition B being packaged in one or more spray devices.

Through the use of this process, coloured coatings are obtained on keratin fibres such as the hair which result in colouring that is visible on all types of hair, i.e. coloured coatings which effectively cover and mask the initial hair colour. Specifically, the process according to the invention allows good coverage of the initial hair colour.

Furthermore, the coloured coating obtained is persistent with respect to shampoo washing and to the various attacking factors to which the keratin fibres such as the hair may be subjected, such as blow-drying and perspiration.

The expression “at least one” means one or more.

Unless otherwise indicated, the limits of a range of values are included in that range, notably in the expressions “between” and “ranging from . . . to . . . ”.

The expression “keratin fibres” is understood particularly to mean human keratin fibres, such as the hair, eyelashes, eyebrows and body hair, preferentially the hair, eyebrows and eyelashes, more preferentially still the hair.

For the purposes of the present invention, the term “colouring that is persistent with respect to shampoo washing” means that the colouring obtained persists after at least one shampoo wash.

For the purposes of the present invention, the term “hair” means head hair. This term does not correspond to body hair, the eyebrows or the eyelashes.

For the purposes of the present invention, the term “silicone” denotes any organosilicon polymer or oligomer of linear or cyclic, branched or crosslinked structure, of variable molecular weight, obtained by polymerization and/or polycondensation of suitably functionalized silanes, and constituted essentially of a repetition of main units in which the silicon atoms are linked together via oxygen atoms (siloxane bond —Si—O—Si—), hydrocarbon-based which are optionally substituted radicals being directly linked via a carbon atom to said silicon atoms.

For the purposes of the present invention and unless otherwise indicated:
an “alkyl” radical denotes a linear or branched saturated radical containing, for example, from 1 to 30 carbon atoms;
an “aminoalkyl” radical denotes an alkyl radical as defined previously, said alkyl radical comprising an NH₂ group;
a “hydroxyalkyl” radical denotes an alkyl radical as defined previously, said alkyl radical comprising an OH group;
an “alkylene” radical denotes a linear or branched divalent saturated C₁-C₁₀ hydrocarbon-based group such as methylene, ethylene or propylene;
a “cycloalkyl” or “alicycloalkyl” radical denotes a cyclic saturated monocyclic or polycyclic, preferably monocyclic, hydrocarbon-based group comprising from 1 to 3 rings, preferably 2 rings, and comprising from 3 to 40 carbon atoms, in particular comprising from 3 to 24 carbon atoms, more particularly from 3 to 20 carbon atoms, even more particularly from 3 to 12 carbon atoms, preferably between 5 and 10 carbon atoms, such as cyclopropyl, cyclopentyl, cyclohexyl, cycloheptyl or norbornyl, or isobornyl, in particular cyclopropyl, cyclopentyl or cyclohexyl, it being understood that the cycloalkyl radical may be substituted with one or more (C₁-C₄)alkyl groups such as methyl; preferably, the cycloalkyl radical is then an isobornyl group;
an “aryl” radical is a monocyclic, bicyclic or tricyclic, fused or non-fused, unsaturated and aromatic hydrocarbon-based cyclic radical, comprising from 6 to 30 carbon atoms, preferably between 6 and 14 carbon atoms, more preferentially between 6 and 12 carbon atoms; preferably, the aryl group comprises 1 ring of 6 carbon atoms such as phenyl, naphthyl, anthryl, phenanthryl and biphenyl, it being understood that the aryl radical may be substituted with one or more (C₁-C₄)alkyl groups such as methyl, preferably tolyl, xylyl, or methylnaphthyl; preferably, the aryl group represents a phenyl;
an “aryloxy” radical denotes an aryl-oxy radical with “aryl” as defined previously;
an “alkoxy” radical denotes an alkyl-oxy radical with “alkyl” as defined previously.

Unless otherwise indicated, when compounds are mentioned in the present patent application, this also includes the optical isomers thereof, the geometric isomers thereof, the tautomers thereof, the salts thereof, alone or as a mixture.

The invention is not limited to the examples illustrated. The characteristics of the various examples may notably be combined within variants which are not illustrated.

### DETAILED DESCRIPTION OF THE INVENTION

### Process According to the Invention

### Composition a

### Alkoxysilane of Formula (I) or of Formula (I′):

Composition A according to the invention comprises at least one alkoxysilane chosen from the compounds of formula (I) or of formula (I′) below, oligomers thereof and/or mixtures thereof:

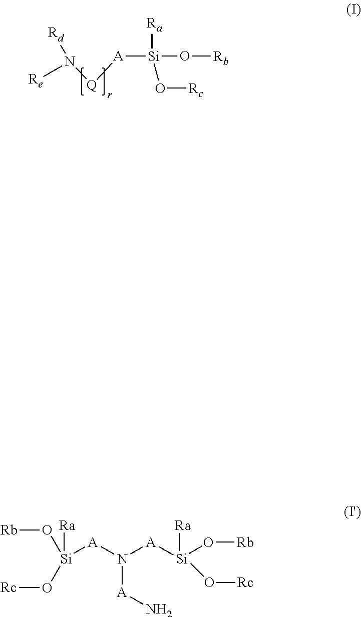

in which:
Rₐ represents an alkyl group containing from 1 to 20 carbon atoms, preferably from 1 to 10 carbon atoms, more preferentially from 1 to 4 carbon atoms and in particular from 1 to 2 carbon atoms such as a methyl, said alkyl group being optionally substituted with an aryl group; an alkoxy group containing from 1 to 10 carbon atoms, preferably from 1 to 4 carbon atoms and in particular from 1 to 2 carbon atoms such as an ethoxy; or an aryl group containing from 6 to 12 carbon atoms;
R_{b} and R_{c}, which may be identical or different, represent a hydrogen atom; an alkyl group containing from 1 to 20 carbon atoms, preferably from 1 to 6 carbon atoms and notably from 1 to 4 carbon atoms, notably an ethyl group, it being understood that if Rₐ does not represent an alkoxy group, then R_{b} and R_{c} cannot simultaneously represent a hydrogen atom;
Rₐ and Rₑ, which may be identical or different, represent a hydrogen atom; an alkyl group containing from 1 to 20 carbon atoms, preferably from 1 to 6 carbon atoms and notably from 1 to 4 carbon atoms; a cycloalkyl group containing from 3 to 20 carbon atoms; an aryl group containing from 6 to 12 carbon atoms; an aminoalkyl group containing from 1 to 20 carbon atoms;
A independently represents a linear or branched alkylene group containing from 1 to 10 carbon atoms, which may be interrupted with at least one heteroatom chosen from O, S, NH or a carbonyl group (CO), preferably NH;
Q represents a carbonyl group (CO);
r denotes an integer ranging from 0 to 1.

Among the alkoxysilanes of formula (I), oligomers thereof and/or mixtures thereof, mention may notably be made of 3-aminopropyltriethoxysilane (APTES), 3-aminopropylmethyldiethoxysilane (APMDES), 3-ureidopropyltrimethoxysilane and N-cyclohexylaminomethyltriethoxysilane.

APTES may be purchased, for example, from the company Dow Corning under the name Xiameter OFS-6011 Silane or from the company Momentive Performance Materials under the name Silsoft A-1100 or from the company Shin-Etsu under the name KBE-903.

The compounds of formula (I) may also denote Dynasylan SIVO 210 or Dynasylan 1505 sold by the company Evonik.

3-Ureidopropyltrimethoxysilane may be purchased, for example, from the company Gelest under the reference SIU9058.0.

N-Cycloheylaminomethyltriethoxysilane may be purchased, for example, from the company Wacker under the name Geniosil XL 926.

Among the alkoxysilanes of formula (I′), oligomers thereof and/or mixtures thereof, mention may notably be made of N,N-bis[3-(trimethoxysilyl)propyl]ethylenediamine (CAS RN: 74956-86-8), N1,N1-bis[3-(triethoxysilyl)propyl]-1,2-ethanediamine (CAS RN: 457065-96-2), 1,2-ethanediamine, N1-[3-(triethoxysilyl)propyl]-N1-[3-(trimethoxysilyl)propyl]-(CAS RN: 1638528-78-5), and mixtures thereof.

Preferably, the alkoxysilane(s), oligomers thereof and/or mixtures thereof are chosen from the compounds of formula (I) below:

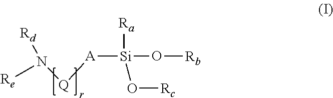

in which:
Rₐ represents an alkyl group containing from 1 to 10 carbon atoms, notably from 1 to 4 carbon atoms and in particular from 1 to 2 carbon atoms, preferably a methyl, or an alkoxy group containing from 1 to 4 carbon atoms, preferably from 1 to 2 carbon atoms, preferably an ethoxy;
R_{b} and R_{c}, which may be identical or different, represent an alkyl group containing from 1 to 10 carbon atoms, preferably from 1 to 4 carbon atoms, such as an ethyl;
Rₐ and Rₑ, which are identical, represent a hydrogen atom or Rₐ denotes a hydrogen atom and Rₑ denotes a C₅-C₆ cycloalkyl radical such as cyclohexyl;
A independently represents a linear or branched alkylene group containing from 1 to 10 carbon atoms, which may be interrupted with at least one heteroatom chosen from O, S and NH or a carbonyl group (CO), preferably NH;
r denotes an integer equal to 0.

Preferably, the alkoxysilane(s), oligomers thereof and/or mixtures thereof are chosen from the compounds of formula (I) in which Rₐ represents an ethoxy group, R_{b} and R_{c} are identical and represent an ethyl, Rₐ and Rₑ represent a hydrogen atom, A represents a propylene and r denotes an integer equal to 0.

According to a preferred embodiment, the alkoxysilane of formula (I), oligomers thereof and/or mixtures thereof, is 3-aminopropyltriethoxysilane (APTES).

The alkoxysilane(s) of formula (I) or of formula (I′), oligomers thereof and/or mixtures thereof may be present in a total amount ranging from 0.1 to 40% by weight, preferably from 0.5 to 30% by weight, preferentially from 0.75 to 25% by weight, better still from 1 to 20% by weight and even better still from 1.5 to 15% by weight, relative to the total weight of composition A.

According to one preferred embodiment, the alkoxysilane(s) of formula (I), oligomers thereof and/or mixtures thereof is (are) present in a total amount ranging from 0.1 to 40% by weight, preferably from 0.5 to 30% by weight, preferentially from 0.75 to 25% by weight, better still from 1 to 20% by weight and even better still from 1.5 to 15% by weight, relative to the total weight of composition A.

### Alkoxysilane of Formula (II):

Composition A according to the invention comprises at least one alkoxysilane of formula (II) below, oligomers thereof and/or mixtures thereof:

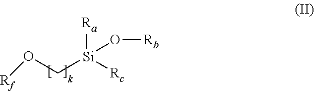

in which:
Rₐ represents an alkyl group containing from 1 to 20 carbon atoms, preferably from 1 to 10 carbon atoms, more preferentially from 1 to 4 carbon atoms and in particular from 1 to 2 carbon atoms such as a methyl, said alkyl group being optionally substituted with an aryl group; an alkoxy group containing from 1 to 10 carbon atoms, preferably from 1 to 4 carbon atoms and in particular from 1 to 2 carbon atoms such as an ethoxy; or an aryl group containing from 6 to 12 carbon atoms;
R_{b} represents a hydrogen atom or an alkyl group containing from 1 to 20 carbon atoms, preferably from 1 to 6 carbon atoms and notably from 1 to 4 carbon atoms, notably an ethyl group;
R_{c} represents an alkyl group containing from 1 to 20 carbon atoms, preferably from 1 to 10 carbon atoms, more preferentially from 1 to 4 carbon atoms and in particular from 1 to 2 carbon atoms such as a methyl, said alkyl group being optionally substituted with an aryl group; an alkoxy group containing from 1 to 10 carbon atoms, preferably from 1 to 4 carbon atoms and in particular from 1 to 2 carbon atoms such as an ethoxy; or an aryl group containing from 6 to 12 carbon atoms;
it being understood that if Rₐ and R_{c} do not represent an alkoxy group, then R_{b} cannot represent a hydrogen atom;
k denotes an integer ranging from 0 to 5, preferably ranging from 0 to 3;
R_{f} represents a hydrogen atom; an alkyl group containing from 1 to 10 carbon atoms and notably from 1 to 4 carbon atoms; or a group of formula (IIa) below:

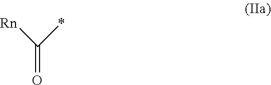

in which Rₙ represents a hydroxyl group (OH); an alkyl group containing from 1 to 10 carbon atoms, preferably a methyl.

Among the alkoxysilanes of formula (II), oligomers thereof and/or mixtures thereof, mention may notably be made of tetraetboxysilane (TEOS), methyltrimethoxysilane (MTMS), methyltriethoxysilane (MTES), dimethyldiethoxysilane (DMDES), diethyldiethoxysilane, dipropyldiethoxysilane, propyltriethoxysilane, isobutyltriethoxysilane, phenyltriethoxysilane, phenylmethyldiethoxysilane, diphenyldiethoxysilane, benzyltriethoxysilane, benzylmethyldiethoxysilane, dibenzyldiethoxysilane, acetoxymethyltriethoxysilane and mixtures thereof.

TEOS may be purchased, for example, from the company Evonik under the name Dynasylan® A or Dynasylan® A SQ.

MTES may be purchased, for example, from the company Evonik under the name Dynasylan® MTES.

DMDES may be purchased, for example, from the company Gelest under the reference SID3404.0.

Preferably, the alkoxysilane(s) of formula (II), oligomers thereof and/or mixtures thereof are such that:
Rₐ represents an alkoxy group containing from 1 to 10 carbon atoms, preferably from 1 to 4 carbon atoms and in particular from 1 to 2 carbon atoms such as a methoxy or an ethoxy; or an alkyl group containing from 1 to 10 carbon atoms optionally substituted with an aryl group, preferably 1 to 2 carbon atoms optionally substituted with an aryl group;
R_{b} represents an alkyl group containing from 1 to 10 carbon atoms, preferably from 1 to 4 carbon atoms, in particular from 1 to 2 carbon atoms, such as a methyl or an ethyl;
R_{c} represents an alkoxy group containing from 1 to 10 carbon atoms, preferably from 1 to 4 carbon atoms and in particular from 1 to 2 carbon atoms, such as a methoxy or an ethoxy;
k denotes an integer ranging from 0 to 3, preferably equal to 0;
R_{f} represents a hydrogen atom or an alkyl group containing from 1 to 10 carbon atoms and notably from 1 to 4 carbon atoms, such as a methyl or an ethyl.

More preferentially, the alkoxysilane(s) of formula (II), oligomers thereof and/or mixtures thereof are such that:
Rₐ represents an alkyl group containing from 1 to 10 carbon atoms, preferably from 1 to 4 carbon atoms, in particular from 1 to 2 carbon atoms, such as a methyl or an ethyl;
R_{b} represents an alkyl group containing from 1 to 10 carbon atoms, preferably from 1 to 4 carbon atoms, in particular from 1 to 2 carbon atoms, such as a methyl or an ethyl;
R_{c} represents an alkoxy group containing from 1 to 10 carbon atoms, preferably from 1 to 4 carbon atoms and in particular from 1 to 2 carbon atoms, such as a methoxy or an ethoxy;
k denotes an integer equal to 0;
R_{f} represents an alkyl group containing from 1 to 10 carbon atoms and notably from 1 to 4 carbon atoms, such as a methyl or an ethyl.

According to a preferred embodiment, the alkoxysilane of formula (II), oligomers thereof and/or mixtures thereof, is methyltrimethoxysilane (MTMS) or methyltriethoxysilane (MTES). The alkoxysilane(s) of formula (II), oligomers thereof and/or mixtures thereof may be present in a total amount ranging from 0.5 to 90% by weight, preferably from 1 to 75%, preferentially from 3 to 45% by weight, better still from 5 to 40% by weight, relative to the total weight of composition A.

### Organic Solvents:

Composition A according to the invention may comprise one or more organic solvents.

Examples of organic solvents that may be mentioned include C₁-C₄ lower alkanols, such as ethanol and isopropanol; polyols and polyol ethers, for instance glycerol, 2-butoxyethanol, propylene glycol, propylene glycol monomethyl ether and diethylene glycol monoethyl ether and monomethyl ether, and also aromatic alcohols, for instance benzyl alcohol or phenoxyethanol, and mixtures thereof.

Preferably, composition A according to the invention comprises ethanol.

The organic solvent(s) may be present in a total amount ranging from 1 to 70% by weight, preferably from 5 to 55% by weight, more preferentially from 10 to 50% by weight, relative to the total weight of composition A according to the invention.

### Composition B

### Film-Forming Polymer:

Composition B according to the invention comprises at least one film-forming polymer.

Preferably, the film-forming polymer(s) are chosen from hydrophilic film-forming polymers, hydrophobic film-forming polymers, and mixtures thereof.

For the purposes of the invention, the term “polymer” means a compound corresponding to the repetition of one or more units (these units being derived from compounds known as monomers). This or these unit(s) are repeated at least twice and preferably at least three times.

The term “hydrophobic polymer” is intended to mean a polymer having a solubility in water at 25° C. of less than 1% by weight.

The term “hydrophilic polymer” is intended to mean a polymer having a solubility in water at 25° C. of greater than or equal to 1% by weight.

The term “film-forming” polymer is intended to mean a polymer that is capable of forming, by itself or in the presence of an auxiliary film-forming agent, a macroscopically continuous film on a support, notably on keratin materials, and preferably a cohesive film.

According to a preferred embodiment, the film-forming polymer(s) are chosen from hydrophobic film-forming polymers.

In a particularly preferred embodiment, the hydrophobic film-forming polymer is a polymer chosen from the group comprising:
film-forming polymers that are soluble in an organic solvent medium, in particular liposoluble polymers; this means that the polymer is soluble or miscible in the organic medium and forms a single homogeneous phase when it is incorporated into the medium;
film-forming polymers that are dispersible in an organic solvent medium, which means that the polymer forms an insoluble phase in the organic medium, the polymer remaining stable and/or compatible once incorporated into this medium. In particular, such polymers may be in the form of non-aqueous dispersions of polymer particles, preferably dispersions in silicone oils or hydrocarbon-based oils; in one embodiment, the non-aqueous polymer dispersions comprise polymer particles stabilized on their surface with at least one stabilizer; these non-aqueous dispersions are often referred to as NADs;
film-forming polymers in the form of aqueous dispersions of polymer particles, which means that the polymer forms an insoluble phase in water, the polymer remaining stable and/or compatible once incorporated into the water, the polymer particles possibly being stabilized on their surface with at least one stabilizer. These polymer particles are often referred to as latexes; in this case, the composition must comprise an aqueous phase.

Among the hydrophobic film-forming polymers that may be used in the composition of the present invention, mention may be made of synthetic polymers, of radical type or of polycondensate type, polymers of natural origin, and mixtures thereof.

Hydrophobic film-forming polymers that may be mentioned in particular include acrylic polymers, polyurethanes, polyesters, polyamides, polyureas, cellulose-based polymers such as nitrocellulose, silicone polymers, polymers of acrylamide type, and polyisoprenes.

Non-ionic, amphoteric, anionic or cationic hydrophobic film-forming polymers may be used.

Preferably, the hydrophobic film-forming polymer(s) according to the invention are chosen from acrylic acid copolymers, methacrylic acid copolymers, acrylic acid ester homopolymers or copolymers, methacrylic acid ester homopolymers or copolymers, acrylic acid amide homopolymers or copolymers, methacrylic acid amide homopolymers or copolymers, vinylpyrrolidone copolymers, vinyl alcohol copolymers, vinyl acetate copolymers, ethylene homopolymers or copolymers, propylene homopolymers or copolymers, styrene homopolymers or copolymers, polyurethanes, polyesters and/or polyamides.

The hydrophobic film-forming polymer can be chosen from homopolymers and copolymers of olefins such as cyclic olefins; of butadiene; of isoprene; of styrene; of vinyl ethers, esters or amides; of (meth)acrylic acid esters or amides containing a linear, branched or cyclic C₁-C₂₀ alkyl group, a C₆-C₁₀ aryl group or a C₂-C₆ hydroxyalkyl group.

The hydrophobic film-forming polymer may notably be chosen from homopolymers and copolymers which may be obtained from monomers chosen from the group constituted of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, ethyl (meth)acrylate, methyl (meth)acrylate, tert-butyl (meth)acrylate, tridecyl (meth)acrylate, stearyl (meth)acrylate, hydroxyethyl (meth)acrylate, 2-hydroxypropyl (meth)acrylate, benzyl acrylate and phenyl acrylate, or mixtures thereof.

Mention may be made, for example, of the ethylene/sodium acrylate copolymer sold under the trade name Ecosmooth Satin® by the company Dow.

The hydrophobic film-forming polymer may notably be chosen from the homopolymers and copolymers that may be obtained from amides of acid monomers; mention may be made of (meth)acrylamides, and notably N-alkyl(meth)acrylamides, in particular of C₂-C₁₂ alkyl, such as N-ethylacrylamide, N-t-butylacrylamide or N-octylacrylamide; N—(C₁-C₄)dialkyl(meth)acrylamides and perfluoroalkyl (meth)acrylates.

Mention may also be made of the copolymers of which the CTFA (4th Ed., 1991) name is Octylacrylamide/Acrylates/Butylaminoethyl Methacrylate Copolymer, such as the products sold under the name Amphomer® or Lovocryl® 47 by the company National Starch, and also the copolymers with the CTFA name Acrylates/Octylacrylamide Copolymer, such as the products sold under the name Dermacryl® LT or Dermacryl® 79 by the company National Starch.

According to a preferred embodiment, the hydrophobic film-forming polymer(s) according to the invention are chosen from anionic copolymers.

Preferably, the anionic copolymers according to the invention are copolymers of acrylic acid, of methacrylic acid or of (meth)acrylic acid esters containing a linear, branched or cyclic C₁-C₆ alkyl group, as described under the INCI name Acrylates.

Such copolymers are sold by the company Rohm & Haas under the name Aculyn®33.

Copolymers of unsaturated ethylenic acid esters of alkoxylated fatty alcohols may also be used according to the invention. Such unsaturated ethylenic acid esters are in particular of acrylic acid, methacrylic acid and itaconic acid, and such alkoxylated fatty alcohols are in particular steareth-20 and ceteth-20.

Mention will be made for example of Aculyn®22 (Acrylates/Steareth-20 Methacrylate Copolymer), Aculyn®28 (Acrylates/Beheneth-25 Methacrylate Copolymer), Structure 2001® (Acrylates/Steareth-20 Itaconate Copolymer), Structure 3001® (Acrylates/Ceteth-20 Itaconate Copolymer), Structure Plus®α(Acrylates/Aminoacrylates/C10-30 Alkyl PEG-20 Itaconate Copolymer), Carbopol®1342, 1382, Ultrez 20, Ultrez 21 (Acrylates/C10-30 Alkyl Acrylate Crosspolymer), Synthalen W2000® (Acrylates/Palmeth-25 Acrylate Copolymer) or Soltex OPT (Acrylates/C₁₂₋₂₂ Alkyl Methacrylate Copolymer) sold by Rohm and Haas.

The hydrophobic film-forming polymer may also be chosen from homopolymers and copolymers that may be obtained from vinyl monomers. Mention may be made of homopolymers or copolymers of N-vinylpyrrolidone, vinylcaprolactam, vinyl-N—(C1-C6)alkylpyrroles, vinyloxazoles, vinylthiazoles, vinylpyrimidines or vinylimidazoles.

As examples of vinylpyrrolidone copolymers that may be used in the invention, mention may be made of the VP/vinyl laurate copolymer, the VP/vinyl stearate copolymer, the butylated polyvinylpyrrolidone (PVP) copolymer, the VP/hexadecene copolymer sold by ISP under the name Ganex V216, the VP/eicosene copolymer sold by ISP under the name Ganex V220, the VP/triacontene copolymer or the VP/acrylic acid/lauryl methacrylate copolymer.

The hydrophobic film-forming polymer may also be chosen from homopolymers and copolymers that may be obtained from olefins such as ethylene, propylene, butenes, isoprene, butadienes.

In one embodiment, the hydrophobic film-forming polymer according to the invention is a block copolymer comprising at least one block constituted of styrene units or styrene derivatives (for example methylstyrene, chlorostyrene or chloromethylstyrene). The copolymer comprising at least one styrene block may be a diblock or triblock copolymer, or even a multiblock, star or radial copolymer. The copolymer comprising at least one styrene block may also comprise, for example, an alkylstyrene (AS) block, an ethylene/butylene (EB) block, an ethylene/propylene (EP) block, a butadiene (B) block, an isoprene (I) block, an acrylate (A) block or a methacrylate (MA) block, or a combination of these blocks. The copolymer comprising at least one block constituted of styrene units or styrene derivatives can be a diblock or triblock copolymer, and in particular of the polystyrene/polyisoprene or polystyrene/polybutadiene type, such as those sold or manufactured under the name Luvitol HSB by BASF SE.

Preferably, the hydrophobic film-forming polymer(s) according to the invention are chosen from vinylpyrrolidone (co)polymers, vinyl alcohol (co)polymers, vinyl acetate (co)polymers, carboxyvinyl (co)polymers, acrylic acid (co)polymers, methacrylic acid (co)polymers, acrylic acid ester (co)polymers, ethylene (co)polymers, acrylamide (co)polymers and mixtures thereof.

More preferentially, the hydrophobic film-forming polymer(s) are chosen from polyvinylpyrrolidone copolymers, acrylamide copolymers, acrylic acid ester homopolymers or copolymers, ethylene homopolymers or copolymers and mixtures thereof.

According to another preferred embodiment, the film-forming polymer(s) are chosen from hydrophilic film-forming polymers.

Non-ionic, anionic or cationic hydrophilic film-forming polymers may be used.

The hydrophilic film-forming polymer may be chosen from vinylpyrrolidone (co)polymers, vinyl alcohol (co)polymers, vinyl acetate polymers, carboxyvinyl (co)polymers, acrylic acid (co)polymers, methacrylic acid (co)polymers, natural gums, polysaccharides and/or acrylamide (co)polymers.

Preferably, the hydrophilic film-forming polymer is chosen from vinylpyrrolidone homopolymers (PVP) and/or vinylpyrrolidone copolymers, more preferentially vinylpyrrolidone homopolymers (PVP).

Examples that may be mentioned include the vinylpyrrolidone homopolymers (PVP) sold under the name Luviskol® K by the company BASF SE, in particular Luviskol® K 90 or Luviskol® K 85 by the company BASF SE.

The polymer PVP K30 sold by the company Ashland Inc. (ISP, POI Chemical) may also be used. PVP K30 is a polyvinylpyrrolidone polymer that is soluble in cold water, having the CAS number 9003-39-8 and a molecular weight of 40 000 g/mol.

Other vinylpyrrolidone homopolymers suitable for the invention are sold under the trade name Luvitec K 17, Luvitec K 30, Luvitec K 60, Luvitec K 80, Luvitec K 85, Luvitec K 90 and Luvitec K 115 by the company BASF SE.

Mention may also be made of the vinylpyrrolidone/vinyl ester copolymers sold under the name Luviskol® by the company BASF SE, in particular the non-ionic polymers Luviskol® VA64 and Luviskol® VA73 (vinylpyrrolidone/vinyl acetate copolymers).

Mention may also be made of styrene/vinylpyrrolidone copolymers, vinylpyrrolidone/vinyl acetate copolymers, vinylpyrrolidone/DMAPA acrylates copolymers and vinylpyrrolidone/vinylcaprolactam/DMAPA acrylates copolymers.

Vinylpyrrolidone/vinylcaprolactam/DMAPA acrylates copolymers may be sold by the company Ashland Inc. under the trade name Aquaflex® SF-40.

The vinylpyrrolidone/DMAPA acrylates copolymers may be sold by the company Ashland Inc. under the trade name Styleze CC-10.

As vinylpyrrolidone copolymers, mention may be made of copolymers obtained by reaction of N-vinylpyrrolidone with at least one monomer chosen from N-vinylformamide, vinyl acetate, ethylene, propylene, acrylamide and vinylcaprolactam.

According to a preferred embodiment, the hydrophilic film-forming polymer(s) are chosen from vinylpyrrolidone homopolymers (PVP), vinylpyrrolidone/vinyl acetate copolymers, vinylpyrrolidone/styrene copolymers, vinylpyrrolidone/ethylene copolymers, vinylpyrrolidone/propylene copolymers, vinylpyrrolidone/vinylcaprolactam copolymers, vinylpyrrolidone/vinylformamide copolymers, vinylpyrrolidone/vinyl alcohol copolymers, and mixtures thereof.

Preferably, the film-forming polymer(s) are chosen from vinylpyrrolidone homopolymers (PVP), vinylpyrrolidone/vinyl acetate copolymers, vinylpyrrolidone/styrene copolymers, vinylpyrrolidone/ethylene copolymers, vinylpyrrolidone/propylene copolymers, vinylpyrrolidone/vinyl caprolactam copolymers, vinylpyrrolidone/vinylformamide copolymers, vinylpyrrolidone/vinyl alcohol copolymers, acrylamide copolymers, acrylic acid ester homopolymers or copolymers, ethylene homopolymers or copolymers, and mixtures thereof.

More preferentially, the film-forming polymer(s) are chosen from vinylpyrrolidone homopolymers (PVP), acrylamide copolymers, acrylic acid ester homopolymers or copolymers, ethylene homopolymers or copolymers, and mixtures thereof.

The film-forming polymer(s) may be present in a total amount ranging from 0.1 to 30% by weight, preferably from 0.5 to 25% by weight and better still from 1 to 20% by weight, relative to the total weight of composition B.

Composition A and/or B

### Non-Associative Non-Ionic Cellulose-Based Polymer

Composition A and/or composition B according to the invention may comprise at least one non-ionic, preferably non-associative, cellulose-based polymer.

The non-associative non-ionic cellulose-based polymer is different from the film-forming polymers mentioned previously.

According to the invention, the term “cellulose-based polymer” means any polysaccharide polymer having, in its structure, sequences of glucose residues joined together via beta-1,4 bonds.

According to the invention, the term “non-associative cellulose-based polymer” means that the cellulose-based polymers do not include any C₈-C₃₀ fatty chains.

The non-associative non-ionic cellulose-based polymers may be chosen from (C₁-C₄)alkylcelluloses, such as methylcelluloses and ethylcelluloses (for example Ethocel Standard 100 Premium from Dow Chemical); hydroxy(C₁-C₄)alkylcelluloses, such as hydroxymethylcelluloses, hydroxyethylcelluloses and hydroxypropylcelluloses; mixed hydroxy(C₁-C₄)alkyl(C₁-C₄)alkylcelluloses, such as hydroxypropylmethylcelluloses (for example Methocel E4M from Dow Chemical), hydroxyethylmethylcelluloses, hydroxyethylethylcelluloses (for example Bermocoll E 481 FQ from AkzoNobel), and hydroxybutylmethylcelluloses, and also mixtures thereof.

Preferably, the non-associative non-ionic cellulose-based polymer(s) are chosen from hydroxy(C₁-C₄)alkylcelluloses.

More preferentially, the non-associative non-ionic cellulose-based polymer(s) are hydroxyethylcellulose and/or hydroxypropylcellulose (HPC).

More preferentially, composition A and/or composition B comprise(s) hydroxyethylcellulose and/or hydroxypropylcellulose (HPC).

In particular, mention may be made of the hydroxyethylcellulose sold by the company Ashland Inc. under the trade name Natrosol 250 HRR PC and the hydroxypropylcellulose sold by the company Ashland Inc. under the trade name Klucel ME Pharm Hydroxypropylcellulose.

The non-ionic cellulose-based polymer(s) may be present in a total amount preferably ranging from 0.01 to 10% by weight, preferentially from 0.05 to 5% by weight and better still from 0.1 to 3% by weight relative to the total weight of composition A and/or composition B.

The non-associative non-ionic cellulose-based polymer(s) may be present in a total amount preferably ranging from 0.01 to 10% by weight, preferentially from 0.05 to 5% by weight and better still from 0.1 to 3% by weight relative to the total weight of composition A and/or composition B.

### Colouring Agent:

Composition A and/or composition B according to the invention comprise(s) at least one colouring agent chosen from pigments, direct dyes and mixtures thereof.

Preferably, composition A according to the invention comprises at least one colouring agent chosen from pigments, direct dyes and mixtures thereof.

Preferably, composition A and/or composition B according to the invention comprise(s) one or more pigments.

Preferably, the colouring agent(s) are chosen from pigments.

The term “pigment” is understood to mean any pigment that gives colour to keratin materials. Their solubility in water at 25° C. and at atmospheric pressure (760 mmHg) is less than 0.05% by weight, and preferably less than 0.01% by weight.

The pigments that may be used are notably chosen from the organic and/or mineral pigments known in the art, notably those described in Kirk-Othmer's Encyclopedia of Chemical Technology and in Ullmann's Encyclopedia of Industrial Chemistry.

They may be natural, of natural origin, or non-natural.

These pigments may be in pigment powder or paste form. They may be coated or uncoated.

The pigments may be chosen, for example, from mineral pigments, organic pigments, lakes, special effect pigments such as nacres or glitter flakes, and mixtures thereof.

The pigment may be a mineral pigment. The term “mineral pigment” refers to any pigment that satisfies the definition in Ullmann's encyclopedia in the chapter on inorganic pigments. Among the mineral pigments that are useful in the present invention, mention may be made of iron oxides, chromium oxides, manganese violet, ultramarine blue, chromium hydrate, ferric blue, titanium oxide and stannic oxide (tin oxide).

The pigment may be an organic pigment. The term “organic pigment” refers to any pigment that satisfies the definition in Ullmann's encyclopedia in the chapter on organic pigments.

The organic pigment may notably be chosen from nitroso, nitro, azo, xanthene, pyrene, quinoline, anthraquinone, triphenylmethane, fluorane, phthalocyanine, metal-complex, isoindolinone, isoindoline, quinacridone, perinone, perylene, diketopyrrolopyrrole, indigo, thioindigo, dioxazine, triphenylmethane and quinophthalone compounds.

In particular, the white or coloured organic pigments may be chosen from carmine, carbon black, aniline black, azo yellow, quinacridone, phthalocyanine blue, the blue pigments codified in the Color Index under the references CI 42090, 69800, 69825, 74100, 74160, the yellow pigments codified in the Color Index under the references CI 11680, 11710, 19140, 20040, 21100, 21108, 47000, 47005, the green pigments codified in the Color Index under the references CI 61565, 61570, 74260, the orange pigments codified in the Color Index under the references CI 11725, 45370, 71105, the red pigments codified in the Color Index under the references CI 12085, 12120, 12370, 12420, 12490, 14700, 15525, 15580, 15620, 15630, 15800, 15850, 15865, 15880, 26100, 45380, 45410, 58000, 73360, 73915, 75470, the pigments obtained by oxidative polymerization of indole or phenol derivatives as described in patent FR 2 679 771.

Examples that may also be mentioned include pigment pastes of organic pigments, such as the products sold by the company Hoechst under the names:
Cosmenyl Yellow 10G: Yellow 3 pigment (CI 11710);
Cosmenyl Yellow G: Yellow 1 pigment (CI 11680);
Cosmenyl Orange GR: Orange 43 pigment (CI 71105);
Cosmenyl Red R: Red 4 pigment (CI 12085);
Cosmenyl Carmine FB: Red 5 pigment (CI 12490);
Cosmenyl Violet RL: Violet 23 pigment (CI 51319);
Cosmenyl Blue A2R: Blue 15.1 pigment (CI 74160);
Cosmenyl Green GG: Green 7 pigment (CI 74260);
Cosmenyl Black R: Black 7 pigment (CI 77266).

The pigments in accordance with the invention may also be in the form of composite pigments, as described in patent EP 1 184 426. These composite pigments may notably be composed of particles comprising an inorganic core, at least one binder for attaching the organic pigments to the core, and at least one organic pigment which at least partially covers the core.

The organic pigment may also be a lake. The term “lake” refers to dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate and aluminium.

Among the dyes, mention may be made of carminic acid. Mention may also be made of the dyes known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), D&C Blue 1 (CI 42 090).

An example of a lake that may be mentioned is the product known under the following name: D&C Red 7 (CI 15 850:1).

The pigment may also be a special effect pigment. The term “special effect pigments” means pigments that generally create a coloured appearance (characterized by a certain shade, a certain vivacity and a certain level of luminance) that is non-uniform and that changes as a function of the conditions of observation (light, temperature, angles of observation, etc.). They thereby differ from coloured pigments, which afford a standard uniform opaque, semi-transparent or transparent shade.

Several types of special effect pigments exist: those with a low refractive index, such as fluorescent or photochromic pigments, and those with a higher refractive index, such as nacres, interference pigments or glitter flakes.

Examples of special effect pigments that may be mentioned include nacreous pigments such as mica covered with titanium or with bismuth oxychloride, coloured nacreous pigments such as mica covered with titanium and with iron oxides, mica covered with iron oxide, mica covered with titanium and notably with ferric blue or with chromium oxide, mica covered with titanium and with an organic pigment as defined previously, and also nacreous pigments based on bismuth oxychloride. Nacreous pigments that may be mentioned include the nacres Cellini sold by BASF (mica-TiO₂-lake), Prestige sold by Eckart (mica-TiO₂), Prestige Bronze sold by Eckart (mica-Fe₂O₃), Syncrystal sold by Eckart (Mica-TiO₂—SnO₂), and Colorona sold by Merck (mica-TiO₂—Fe₂O₃).

Mention may also be made of the gold-coloured nacres sold notably by the company BASF under the name Brilliant Gold 212G (Timica), Gold 222C (Cloisonne), Sparkle Gold (Timica), Gold 4504 (Chromalite) and Monarch Gold 233X (Cloisonne); the bronze nacres sold notably by the company Merck under the name Bronze Fine (17384) (Colorona) and Bronze (17353) (Colorona) and by the company BASF under the name Super Bronze (Cloisonne); the orange nacres sold notably by the company BASF under the name Orange 363C (Cloisonne) and Orange MCR 101 (Cosmica) and by the company Merck under the name Passion Orange (Colorona) and Matte Orange (17449) (Microna); the brown nacres sold notably by the company BASF under the name Nu-Antique Copper 340XB (Cloisonne) and Brown CL4509 (Chromalite); the nacres with a copper tint sold notably by the company BASF under the name Copper 340A (Timica); the nacres with a red tint sold notably by the company Merck under the name Sienna Fine (17386) (Colorona); the nacres with a yellow tint sold notably by the company BASF under the name Yellow (4502) (Chromalite); the red nacres with a gold tint sold notably by the company BASF under the name Sunstone G012 (Gemtone); the pink nacres sold notably by the company BASF under the name Tan Opal G005 (Gemtone); the black nacres with a gold tint sold notably by the company BASF under the name Nu-Antique Bronze 240 AB (Timica), the blue nacres sold notably by the company Merck under the name Matte Blue (17433) (Microna), the white nacres with a silvery tint sold notably by the company Merck under the name Xirona Silver, and the golden-green pink-orange nacres sold notably by the company Merck under the name Indian Summer (Xirona), and mixtures thereof.

Still as examples of nacres, mention may also be made of particles including a borosilicate substrate coated with titanium oxide.

Particles comprising a glass substrate coated with titanium oxide are notably sold under the name Metashine MC1080RY by the company Toyal.

Finally, examples of nacres that may also be mentioned include polyethylene terephthalate glitter flakes, notably those sold by the company Meadowbrook Inventions under the name Silver 1P 0.004×0.004 (silver glitter flakes). It is also possible to envisage multilayer pigments based on synthetic substrates, such as alumina, silica, calcium sodium borosilicate, calcium aluminium borosilicate and aluminium.

The special effect pigments may also be chosen from reflective particles, i.e. notably from particles of which the size, structure, notably the thickness of the layer(s) of which they are made and their physical and chemical nature, and surface state, allow them to reflect incident light. This reflection may, where appropriate, have an intensity sufficient to create at the surface of the composition or of the mixture, when it is applied to the support to be made up, highlight points that are visible to the naked eye, i.e. brighter points that contrast with their surroundings, making them appear to sparkle.

The reflective particles may be selected so as not to significantly alter the colouring effect generated by the colouring agents with which they are combined, and more particularly so as to optimize this effect in terms of colour rendition. They may more particularly have a yellow, pink, red, bronze, orange, brown, gold and/or coppery colour or tint.

These particles may have varied forms and may notably be in platelet or globular form, in particular in spherical form.

The reflective particles, whatever their form, may or may not have a multilayer structure and, in the case of a multilayer structure, may have, for example, at least one layer of uniform thickness, notably of a reflective material.

When the reflective particles do not have a multilayer structure, they may be composed, for example, of metal oxides, notably titanium or iron oxides obtained synthetically.

When the reflective particles have a multilayer structure, they may include, for example, a natural or synthetic substrate, notably a synthetic substrate at least partially coated with at least one layer of a reflective material, notably of at least one metal or metallic material. The substrate may be made of one or more organic and/or inorganic materials.

More particularly, it may be chosen from glasses, ceramics, graphite, metal oxides, aluminas, silicas, silicates, notably aluminosilicates and borosilicates, and synthetic mica, and mixtures thereof, this list not being limiting.

The reflective material may include a layer of metal or of a metallic material.

Reflective particles are notably described in JP-A-09188830, JP-A-10158450, JP-A-10158541, JP-A-07258460 and JP-A-05017710.

Again as an example of reflective particles including a mineral substrate coated with a layer of metal, mention may also be made of particles including a silver-coated borosilicate substrate. Particles with a silver-coated glass substrate, in the form of platelets, are sold under the name Microglass Metashine REFSX 2025 PS by the company Toyal. Particles with a glass substrate coated with a nickel/chromium/molybdenum alloy are sold under the names Crystal Star GF 550 and GF 2525 by this same company.

Use may also be made of particles comprising a metal substrate, such as silver, aluminium, iron, chromium, nickel, molybdenum, gold, copper, zinc, tin, magnesium, steel, bronze or titanium, said substrate being coated with at least one layer of at least one metal oxide, such as titanium oxide, aluminium oxide, iron oxide, cerium oxide, chromium oxide, silicon oxides and mixtures thereof.

Examples that may be mentioned include aluminium powder, bronze powder or copper powder coated with SiO₂, sold under the name Visionaire by the company Eckart.

Mention may also be made of interference pigments which are not attached to a substrate, such as liquid crystals (Helicones HC from Wacker) or interference holographic glitter flakes (Geometric Pigments or Spectra f/x from Spectratek). Special effect pigments also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

The variety of pigments that may be used in the present invention makes it possible to obtain a wide range of colours, and also particular optical effects such as metallic effects or interference effects.

The size of the pigment used in the composition according to the present invention is generally between 5 nm and 200 μm, preferably between 7 nm and 80 μm and more preferentially between 10 nm and 50 μm.

The pigments may be dispersed in the composition by means of a dispersant.

The dispersant serves to protect the dispersed particles against agglomeration or flocculation thereof. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. In particular, esters of 12-hydroxystearic acid in particular and of C8 to C20 fatty acid and of polyols such as glycerol or diglycerol are used, such as poly(12-hydroxystearic acid) stearate with a molecular weight of approximately 750 g/mol, such as the product sold under the name Solsperse 21 000 by the company Avecia, polyglyceryl-2 dipolyhydroxystearate (CTFA name) sold under the reference Dehymyls PGPH by the company Henkel, or else polyhydroxystearic acid such as the product sold under the reference Arlacel P100 by the company Uniqema, and mixtures thereof.

As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The pigments used in the composition may be surface-treated with an organic agent.

Thus, the pigments surface-treated beforehand that are useful in the context of the invention are pigments which have been completely or partially subjected to a surface treatment of chemical, electronic, electrochemical, mechanochemical or mechanical nature with an organic agent, such as those described notably in Cosmetics and Toiletries, February 1990, Vol. 105, pages 53-64, before being dispersed in the composition in accordance with the invention. These organic agents may be chosen, for example, from waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof; anionic surfactants; lecithins; sodium, potassium, magnesium, iron, titanium, zinc or aluminium salts of fatty acids, for example aluminium stearate or laurate; metal alkoxides; polyethylene; (meth)acrylic polymers, for example polymethyl methacrylates; polymers and copolymers containing acrylate units; alkanolamines; silicone compounds, for example silicones, notably polydimethylsiloxanes; organofluorine compounds, for example perfluoroalkyl ethers; fluorosilicone compounds.

The surface-treated pigments that are useful in the composition may also have been treated with a mixture of these compounds and/or may have undergone several surface treatments.

The surface-treated pigments that are useful in the context of the present invention may be prepared according to surface-treatment techniques that are well known to those skilled in the art, or may be commercially available as is.

Preferably, the surface-treated pigments are coated with an organic layer.

The organic agent with which the pigments are treated may be deposited on the pigments by evaporation of solvent, chemical reaction between the molecules of the surface agent or creation of a covalent bond between the surface agent and the pigments.

The surface treatment may thus be performed, for example, by chemical reaction of a surface agent with the surface of the pigments and creation of a covalent bond between the surface agent and the pigments or the fillers. This method is notably described in patent U.S. Pat. No. 4,578,266.

An organic agent covalently bonded to the pigments will preferably be used.

The agent for the surface treatment may represent from 0.1% to 50% by weight relative to the total weight of the surface-treated pigment, preferably from 0.5% to 30% by weight and even more preferentially from 1% to 20% by weight relative to the total weight of the surface-treated pigment.

Preferably, the surface treatments of the pigments are chosen from the following treatments:
a PEG-silicone treatment, for instance the AQ surface treatment sold by LCW;
a methicone treatment, for instance the SI surface treatment sold by LCW;
a dimethicone treatment, for instance the Covasil 3.05 surface treatment sold by LCW;
a dimethicone/trimethylsiloxysilicate treatment, for instance the Covasil 4.05 surface treatment sold by LCW;
a magnesium myristate treatment, for instance the MM surface treatment sold by LCW;
an aluminium dimyristate treatment, for instance the MI surface treatment sold by Miyoshi;
a perfluoropolymethyl isopropyl ether treatment, for instance the FHC surface treatment sold by LCW;
an isostearyl sebacate treatment, for instance the HS surface treatment sold by Miyoshi;
a perfluoroalkyl phosphate treatment, for instance the PF surface treatment sold by Daito;
an acrylate/dimethicone copolymer and perfluoroalkyl phosphate treatment, for instance the FSA surface treatment sold by Daito;
a polymethylhydrogenosiloxane/perfluoroalkyl phosphate treatment, for instance the FS01 surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
an acrylate copolymer treatment, for instance the APD surface treatment sold by Daito;
a perfluoroalkyl phosphate/isopropyl titanium triisostearate treatment, for instance the PF+ITT surface treatment sold by Daito.

According to a particular embodiment of the invention, the dispersant is present with organic or inorganic pigments in submicron-sized particulate form in the dye composition.

The term “submicron-sized” or “submicronic” refers to pigments having a particle size that has been micronized by a micronization method and having a mean particle size of less than a micrometre (μm), in particular between 0.1 and 0.9 μm, and preferably between 0.2 and 0.6 μm.

According to one embodiment, the dispersant and the pigment(s) are present in a (dispersant:pigment) amount of between 1:4 and 4:1, particularly between 1.5:3.5 and 3.5:1 or better still between 1.75:3 and 3:1.

The dispersant(s) may thus have a silicone backbone, such as silicone polyether and dispersants of aminosilicone type, different from the alkoxysilanes of formula (I) or formula (I′) described previously in the application. Among the suitable dispersants that may be mentioned are:
aminosilicones, i.e. silicones comprising one or more amino groups such as those sold under the names and references: BYK LPX 21879 by BYK, GP-4, GP-6, GP-344, GP-851, GP-965, GP-967 and GP-988-1, sold by Genesee Polymers,
silicone acrylates such as Tego® RC 902, Tego® RC 922, Tego® RC 1041, and Tego® RC 1043, sold by Evonik,
polydimethylsiloxane (PDMS) silicones bearing carboxyl groups such as X-22162 and X-22370 by Shin-Etsu, epoxy silicones such as GP-29, GP-32, GP-502, GP-504, GP-514, GP-607, GP-682, and GP-695 by Genesee Polymers, or Tego® RC 1401, Tego® RC 1403, Tego® RC 1412 by Evonik.

According to one particular embodiment, the dispersant(s) are of aminosilicone type, different from the alkoxysilanes of formula (I) or formula (I′) described previously in the application and are cationic.

Preferably, the pigment(s) is (are) chosen from mineral, mixed mineral-organic or organic pigments or special effect pigments.

In one variant of the invention, the pigment(s) according to the invention are organic pigments, preferentially organic pigments surface-treated with an organic agent chosen from silicone compounds.

In another variant of the invention, the pigment(s) according to the invention are mineral pigments.

In another variant of the invention, the pigments according to the invention are special effect pigments, preferably nacreous pigments, more preferentially coloured nacreous pigments.

Composition A and/or composition B according to the invention may comprise one or more direct dyes.

The term “direct dye” means natural and/or synthetic dyes, other than oxidation dyes. These are dyes which will spread superficially over the fibre.

They may be ionic or non-ionic, preferably anionic, cationic or non-ionic.

Examples of suitable direct dyes that may be mentioned include azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (III) and (IV) and the azo cationic dyes (V) and (VI) below:

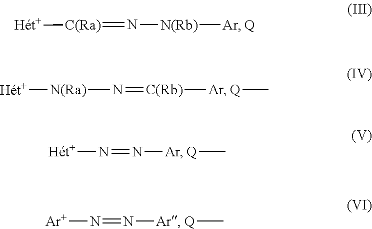

in which formulae (III) to (VI):
Het⁺ represents a cationic heteroaryl radical, preferentially bearing an endocyclic cationic charge, such as imidazolium, indolium or pyridinium, which is optionally substituted, preferentially with at least one (C₁-C₈)alkyl group such as methyl;
Ar⁺ represents an aryl radical, such as phenyl or naphthyl, bearing an exocyclic cationic charge, preferentially ammonium, particularly tri(C₁-C₈)alkylammonium, such as trimethylammonium;
Ar represents an aryl group, notably phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
Rₐ and R_{b}, which may be identical or different, represent a hydrogen atom or a (C₁-C₈)alkyl group which is optionally substituted, preferentially with a hydroxyl group;
or else the substituent Rₐ with a substituent of Het⁺ and/or R_{b} with a substituent of Ar form, together with the atoms that bear them, a (hetero)cycloalkyl; in particular, Ra and Rb represent a hydrogen atom or a (C₁-C₄)alkyl group which is optionally substituted with a hydroxyl group;
Q⁻ represents an organic or mineral anionic counterion, such as a halide or an alkyl sulfate.

In particular, mention may be made of the azo and hydrazono direct dyes bearing an endocyclic cationic charge of formulae (III) to (VI) as defined previously, more particularly the cationic direct dyes bearing an endocyclic cationic charge described in patent applications WO 95/15144, WO 95/01772 and EP 714 954,
Preferentially the following direct dyes:

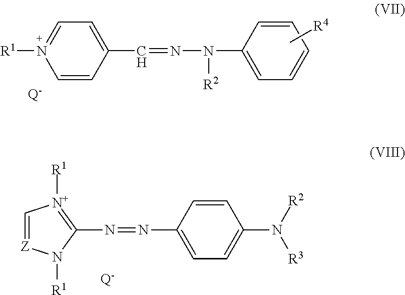

in which formulae (VII) and (VIII):
R¹ represents a (C₁-C₄)alkyl group such as methyl;
R² and R³, which may be identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
Z represents a CH group or a nitrogen atom, preferentially CH,
Q⁻ is an anionic counterion as defined previously, in particular a halide, such as chloride, or an alkyl sulfate, such as methyl sulfate or mesityl.

In particular, the dyes of formulae (VII) and (VIII) are chosen from Basic Red 51, Basic Yellow 87 and Basic Orange 31 or derivatives thereof with Q⁻ being an anionic counterion as defined previously, particularly halide such as chloride, or an alkyl sulfate such as methyl sulfate or mesityl.

The direct dyes may be chosen from anionic direct dyes. The anionic direct dyes of the invention are dyes commonly referred to as “acid” direct dyes owing to their affinity for alkaline substances. The term “anionic direct dye” means any direct dye including in its structure at least one CO₂R or SO₃R substituent with R denoting a hydrogen atom or a cation originating from a metal or an amine, or an ammonium ion. The anionic dyes may be chosen from direct nitro acid dyes, azo acid dyes, azine acid dyes, triarylmethane acid dyes, indoamine acid dyes, anthraquinone acid dyes, indigoid dyes and natural acid dyes.

As acid dyes according to the invention, mention may be made of the dyes of formulae (IX), (IX′), (X), (X′), (XI), (XI′), (XII), (XII′), (XIII), (XIV), (XV) and (XVI) below:
a) the diaryl anionic azo dyes of formula (IX) or (IX′):

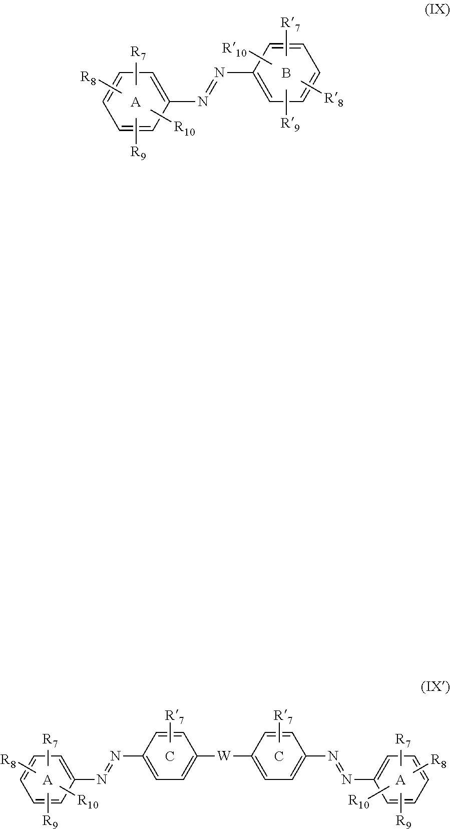

in which formulae (IX) and (IX′):
R₇, R₈, R₉, R₁₀, R′₇, R′₈, R′₉ and R′₁₀, which may be identical or different, represent a hydrogen atom or a group chosen from:
alkyl;
alkoxy, alkylthio;
hydroxyl, mercapto;
nitro, nitroso;
R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ as defined previously;
R″—S(O)₂—, with R″ representing a hydrogen atom or an alkyl group, an aryl, (di)(alkyl)amino or aryl(alkyl)amino group; preferentially a phenylamino or phenyl group;
R′″—S(O)₂—X′— with R′″ representing an alkyl group, aryl group which is optionally substituted, X′ as defined previously;
(di)(alkyl)amino;
aryl(alkyl)amino optionally substituted with one or more groups chosen from i) nitro; ii) nitroso; iii) (O)₂S(O⁻)—, M⁺ and iv) alkoxy with M⁺ as defined previously;
optionally substituted heteroaryl; preferentially a benzothiazolyl group;
cycloalkyl; notably cyclohexyl;
Ar—N═N— with Ar representing an optionally substituted aryl group, preferentially a phenyl optionally substituted with one or more alkyl, (O)₂S(O⁻)—, M⁺ or phenylamino groups;
or alternatively two contiguous groups R₇ with R₈ or R₈ with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R^{o}—C(X)—X′—; viii) R^{o}—X′—C(X)—; ix) R^{o}—X′—C(X)—X″—; x) Ar—N═N— and xi) optionally substituted aryl(alkyl)amino; with M⁺, R^{o}, X, X′, X″ and Ar as defined previously;
W represents a sigma a bond, an oxygen or sulfur atom, or a divalent radical i) —NR— with R as defined previously, or ii) methylene —C(Ra)(Rb)— with Ra and Rb, which may be identical or different, representing a hydrogen atom or an aryl group, or alternatively Ra and Rb form, together with the carbon atom that bears them, a spiro cycloalkyl; preferentially, W represents a sulfur atom or Ra and Rb together form a cyclohexyl;
it being understood that formulae (IX) and (IX′) comprise at least one sulfonate radical (O)₂S(O⁻)—, M⁺ or one carboxylate radical (O)CO⁻—, M⁺ on one of the rings A, A′, B, B′ or C; preferentially sodium sulfonate.

As examples of dyes of formula (IX), mention may be made of: Acid Red 1, Acid Red 4, Acid Red 13, Acid Red 14, Acid Red 18, Acid Red 27, Acid Red 28, Acid Red 32, Acid Red 33, Acid Red 35, Acid Red 37, Acid Red 40, Acid Red 41, Acid Red 42, Acid Red 44, Pigment Red 57, Acid Red 68, Acid Red 73, Acid Red 135, Acid Red 138, Acid Red 184, Food Red 1, Food Red 13, Acid Orange 6, Acid Orange 7, Acid Orange 10, Acid Orange 19, Acid Orange 20, Acid Orange 24, Yellow 6, Acid Yellow 9, Acid Yellow 36, Acid Yellow 199, Food Yellow 3; Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2; Food Yellow 3 or Sunset Yellow; and as examples of dyes of formula (IX′), mention may be made of: Acid Red 111, Acid Red 134, Acid Yellow 38.
b) the pyrazolone anionic azo dyes of formulae (X) and (X′):

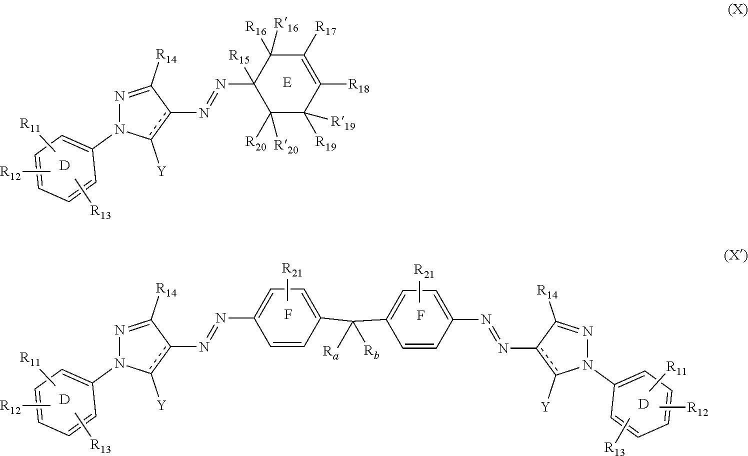

in which formulae (X) and (X′):
R₁₁, R₁₂ and R₁₃, which may be identical or different, represent a hydrogen or halogen atom, an alkyl group or —(O)₂S(O⁻), M⁺ with M⁺ as defined previously;
R₁₄ represents a hydrogen atom, an alkyl group or a group —C(O)O⁻, M⁺ with M⁺ as defined previously;
R₁₅ represents a hydrogen atom;
R₁₆ represents an oxo group, in which case R′₁₆ is absent, or alternatively R₁₅ with R₁₆ together form a double bond;
R₁₇ and R₁₈, which may be identical or different, represent a hydrogen atom, or a group chosen from:
(O)₂S(O⁻)—, M⁺ with M⁺ as defined previously;
Ar—O—S(O)₂— with Ar representing an optionally substituted aryl group; preferentially a phenyl optionally substituted with one or more alkyl groups;
R₁₉ and R₂₀ together form either a double bond, or a benzo group D′ which is optionally substituted;
R′₁₆, R′₁₉ and R′₂₀, which may be identical or different, represent a hydrogen atom or an alkyl or hydroxyl group;
R₂₁ represents a hydrogen atom or an alkyl or alkoxy group;
Ra and Rb, which may be identical or different, are as defined previously; preferentially, Ra represents a hydrogen atom and Rb represents an aryl group;
Y represents either a hydroxyl group or an oxo group;
[symbol] represents a single bond when Y is an oxo group; and represents a double bond when Y represents a hydroxyl group;
it being understood that formulae (X) and (X′) comprise at least one sulfonate radical (O)₂S(O⁻)—, M⁺ or one carboxylate radical —C(O)O⁻, M⁺ on one of the rings D or E; preferentially sodium sulfonate.

As examples of dyes of formula (X), mention may be made of: Acid Red 195, Acid Yellow 23, Acid Yellow 27, Acid Yellow 76, and as examples of dyes of formula (X′), mention may be made of: Acid Yellow 17;
c) the anthraquinone dyes of formulae (XI) and (XI′):

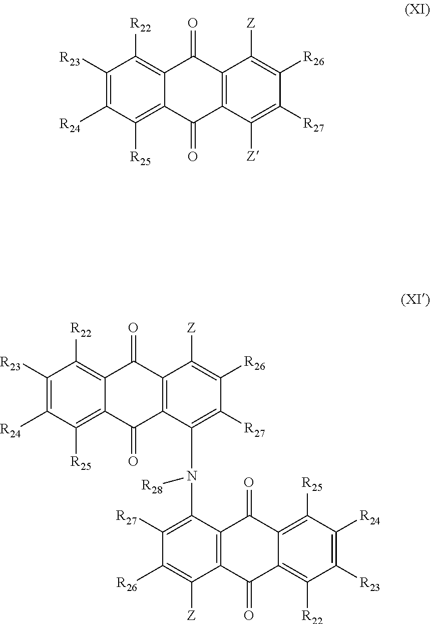

in which formulae (XI) and (XI′):
R₂₂, R₂₃, R₂₄, R₂₅, R₂₆ and R₂₇, which may be identical or different, represent a hydrogen or halogen atom, or a group chosen from:
alkyl;
hydroxyl, mercapto;
alkoxy, alkylthio;
optionally substituted aryloxy or optionally substituted arylthio, preferentially substituted with one or more groups chosen from alkyl and (O)₂S(O⁻)—, M⁺ with M⁺ as defined previously;
aryl(alkyl)amino optionally substituted with one or more groups chosen from alkyl and (O)₂S(O⁻)—, M⁺ with M⁺ as defined previously;
(di)(alkyl)amino;
(di)(hydroxyalkyl)amino;
(O)₂S(O⁻)—, M⁺ with M⁺ as defined previously;
Z′ represents a hydrogen atom or a group NR₂₈R₂₉ with R₂₈ and R₂₉, which may be identical or different, representing a hydrogen atom or a group chosen from:
alkyl;
polyhydroxyalkyl such as hydroxyethyl;
aryl optionally substituted with one or more groups, particularly i) alkyl such as methyl, n-dodecyl, n-butyl; ii) (O)₂S(O⁻)—, M⁺ with M⁺ as defined previously; iii) R^{o}—C(X)—X′—, R^{o}—X′—C(X)—, R^{o}—X′—C(X)—X″— with R^{o}, X, X′ and X″ as defined previously, preferentially R^{o} represents an alkyl group;
cycloalkyl; notably cyclohexyl;
Z represents a group chosen from hydroxyl and NR′₂₈R′₂₉ with R′₂₈ and R′₂₉, which may be identical or different, representing the same atoms or groups as R₂₈ and R₂₉ as defined previously;
it being understood that formulae (XI) and (XI′) comprise at least one sulfonate radical (O)₂S(O⁻)—, M⁺ or one carboxylate radical C(O)O—, M⁺; preferentially sodium sulfonate.

As examples of dyes of formula (XI), mention may be made of: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; EXT Violet No. 2; and, as an example of a dye of formula (XI′), mention may be made of: Acid Black 48; d) the nitro dyes of formulae (XII) and (XII′):

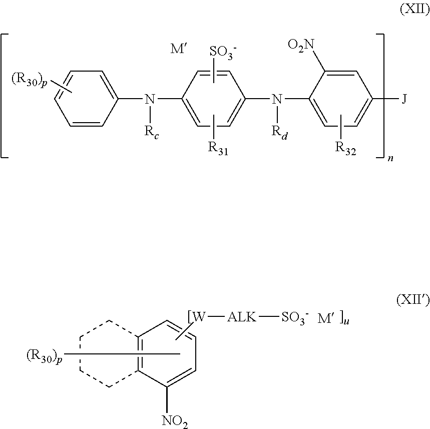

in which formulae (XII) and (XII′):
R₃₀, R₃₁ and R₃₂, which may be identical or different, represent a hydrogen or halogen atom, or a group chosen from:
alkyl;
alkoxy optionally substituted with one or more hydroxyl groups, alkylthio optionally substituted with one or more hydroxyl groups;
hydroxyl, mercapto;
nitro, nitroso;
polyhaloalkyl;
R^{o}—C(X)—X′—, R^{o}—X′—C(X)—, R^{o}—X′—C(X)—X″— with R^{o}, X, X′ and X″ as defined previously;
(O)₂S(O⁻)—, M⁺ with M⁺ as defined previously;
(O)CO⁻—, M⁺ with M⁺ as defined previously;
(di)(alkyl)amino;
(di)(hydroxyalkyl)amino;
heterocycloalkyl such as piperidino, piperazino or morpholino; in particular, R₃₀, R₃₁ and R₃₂ represent a hydrogen atom;
Rc and Rd, which may be identical or different, represent a hydrogen atom or an alkyl group;
W is as defined previously; W particularly represents an —NH— group;
ALK represents a linear or branched divalent C₁-C₆ alkylene group; in particular, ALK represents a —CH₂—CH₂— group;
n is 1 or 2;
p represents an integer between 1 and 5 inclusive;
q represents an integer between 1 and 4 inclusive;
u is 0 or 1;
when n is 1, J represents a nitro or nitroso group; particularly nitro;
when n is 2, J represents an oxygen or sulfur atom, or a divalent radical —S(O)ₘ— with m representing an integer 1 or 2; preferentially, J represents an —SO₂— radical;
M′ represents a hydrogen atom or a cationic counterion;

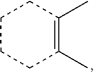

which may be present or absent, represents a benzo group optionally substituted with one or more groups R₃₀ as defined previously;
it being understood that formulae (XII) and (XII′) comprise at least one sulfonate radical (O)₂S(O⁻)—, M⁺ or one carboxylate radical —C(O)O—, M⁺; preferentially sodium sulfonate.

As examples of dyes of formula (XII), mention may be made of: Acid Brown 13 and Acid Orange 3; as examples of dyes of formula (XII′), mention may be made of: Acid Yellow 1, the sodium salt of 2,4-dinitro-1-naphthol-7-sulfonic acid, 2-piperidino-5-nitrobenzenesulfonic acid, 2-(4′-N,N-(2″-hydroxyethyl)amino-2′-nitro)anilineethanesulfonic acid, 4-β-hydroxyethylamino-3-nitrobenzenesulfonic acid; EXT D&C Yellow 7;
e) the triarylmethane dyes of formula (XIII):

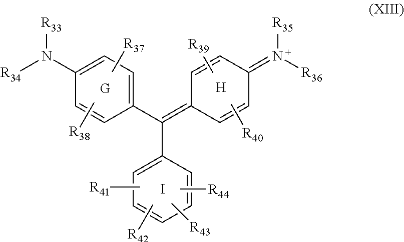

in which formula (XIII):
R₃₃, R₃₄, R₃₅ and R₃₆, which may be identical or different, represent a hydrogen atom or a group chosen from alkyl, optionally substituted aryl and optionally substituted arylalkyl; particularly an alkyl and benzyl group optionally substituted with a group (O)ₘS(O⁻)—, M⁺ with M⁺ and m as defined previously;
R₃₇, R₃₈, R₃₉, R₄₀, R₄₁, R₄₂, R₄₃ and R₄₄, which may be identical or different, represent a hydrogen atom or a group chosen from:
alkyl;
alkoxy, alkylthio;
(di)(alkyl)amino;
hydroxyl, mercapto;
nitro, nitroso;
R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ as defined previously;
or alternatively two contiguous groups R₄₁ with R₄₂ or R₄₂ with R₄₃ or R₄₃ with R₄₄ together form a fused benzo group: I′; with I′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R^{o}—C(X)—X′—; viii) R^{o}—X′—C(X)— and ix) R^{o}—X′—C(X)—X″—; with M⁺, R^{o}, X, X′ and X″ as defined previously;
in particular, R₃₇ to R₄₀ represent a hydrogen atom, and R₄₁ to R₄₄, which may be identical or different, represent a hydroxyl group or (O)₂S(O⁻)—, M⁺; and when R₄₃ with R₄₄ together form a benzo group, it is preferentially substituted with an (O)₂S(O⁻)— group;
it being understood that at least one of the rings G, H, I or I′ comprises at least one sulfonate radical (O)₂S(O⁻)— or a carboxylate radical —C(O)O—; preferentially sulfonate;
As examples of dyes of formula (XIII), mention may be made of: Acid Blue 1; Acid Blue 3; Acid Blue 7, Acid Blue 9; Acid Violet 49; Acid Green 3; Acid Green 5 and Acid Green 50;
f) the xanthene-based dyes of formula (XIV):

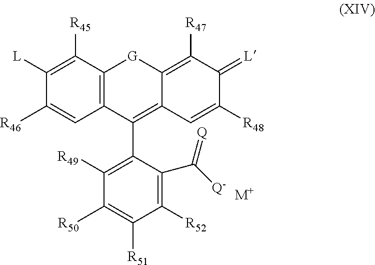

in which formula (XIV):
R₄₅, R₄₆, R₄₇ and R₄₈, which may be identical or different, represent a hydrogen or halogen atom;
R₄₉, R₅₀, R₅₁ and R₅₂, which may be identical or different, represent a hydrogen or halogen atom or a group chosen from:
alkyl;
alkoxy, alkylthio;
hydroxyl, mercapto;
nitro, nitroso;
(O)₂S(O⁻)—, M⁺ with M⁺ representing a hydrogen atom or a cationic counterion;
(O)CO⁻—, M⁺ with M⁺ as defined previously;
particularly R₅₃, R₅₄, R₅₅ and R₄₈ represent a hydrogen or halogen atom;
G represents an oxygen or sulfur atom or a group NRe with Re as defined previously; particularly, G represents an oxygen atom;
L represents an alkoxide O⁻, M⁺; a thioalkoxide S⁻, M⁺ or a group NRf, with Rf representing a hydrogen atom or an alkyl group, and M⁺ as defined previously; M⁺ is particularly sodium or potassium;
L′ represents an oxygen or sulfur atom or an ammonium group: N⁺RfRg, with Rf and Rg, which may be identical or different, representing a hydrogen atom, an alkyl group, aryl group which is optionally substituted; L′ particularly represents an oxygen atom or a phenylamino group optionally substituted with one or more alkyl or (O)ₘS(O⁻)—, M⁺ groups with m and M⁺ as defined previously;
Q and Q′, which may be identical or different, represent an oxygen or sulfur atom; particularly, Q and Q′ represent an oxygen atom;
M⁺ is as defined previously.

As examples of dyes of formula (XIV), mention may be made of: Acid Yellow 73; Acid Red 51; Acid Red 52; Acid Red 87; Acid Red 92; Acid Red 95; Acid Violet 9;
g) the indole-based dyes of formula (XV):

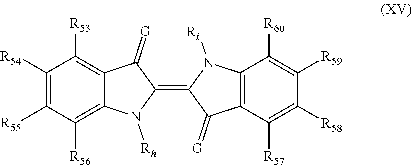

R₅₃, R₅₄, R₅₅, R₅₆, R₅₇, R₅₈, R₅₉ and R₆₀, which may be identical or different, represent a hydrogen atom or a group chosen from:
alkyl;
alkoxy, alkylthio;
hydroxyl, mercapto;
nitro, nitroso;
R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ as defined previously;
G represents an oxygen or sulfur atom or a group NRe with Rₑ as defined previously; particularly, G represents an oxygen atom;
Ri and Rh, which may be identical or different, represent a hydrogen atom or an alkyl group; it being understood that formula (XV) comprises at least one sulfonate radical (O)₂S(O⁻)—, M⁺ or one carboxylate radical —C(O)O—, M⁺; preferentially sodium sulfonate.

As examples of dyes of formula (XV), mention may be made of: Acid Blue 74.
h) the quinoline-based dyes of formula (XVI):

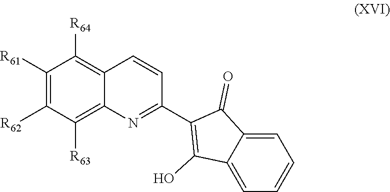

R₆₁ represents a hydrogen or halogen atom or an alkyl group;
R₆₂, R₆₃ and R₆₄, which may be identical or different, represent a hydrogen atom or a group (O)₂S(O⁻)—, M⁺ with M⁺ representing a hydrogen atom or a cationic counterion; or alternatively R₆₁ with R₆₂, or R₆₁ with R₆₄, together form a benzo group optionally substituted with one or more groups (O)₂S(O⁻)—, M⁺ with M⁺ representing a hydrogen atom or a cationic counterion;
it being understood that formula (XVI) comprises at least one sulfonate radical (O)₂S(O⁻)—, M⁺, preferentially sodium sulfonate.

As examples of dyes of formula (XVI), mention may be made of: Acid Yellow 2, Acid Yellow 3 and Acid Yellow 5.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions containing these natural dyes and notably henna-based poultices or extracts.

Preferably, the direct dyes are chosen from anionic direct dyes.

The colouring agent(s) may be present in a total amount ranging from 0.001 to 20% by weight, preferably from 0.005 to 15% by weight and better still from 0.005 to 10% by weight, relative to the total weight of composition A and/or composition B.

The pigment(s) may be present in a total amount ranging from 0.05 to 20% by weight, preferably from 0.1 to 15% by weight and better still from 0.5 to 10% by weight, relative to the total weight of composition A and/or composition B.

The direct dye(s) may be present in a total amount ranging from 0.001 to 10% by weight and preferably from 0.005 to 5% by weight relative to the total weight of composition A and/or composition B.

### Solvents:

Composition A and/or composition B used in the context of the process according to the invention may comprise water.

Preferably, water is present in a content ranging from 0.1 to 99% by weight, more preferentially from 1 to 98% by weight and better still from 10 to 97% by weight relative to the total weight of composition A and/or composition B.

When composition A and/or composition B according to the invention comprise(s) water, the pH of composition A and/or composition B is preferably alkaline, more preferentially the pH of composition A and/or composition B is between 9 and 11.

For the purpose of adjusting the pH, composition A and/or composition B may comprise an alkaline agent.

Preferably, composition A and/or composition B according to the invention comprises an alkaline agent.

The pH of the compositions is measured at room temperature.

As alkaline agent, mention may be made of aqueous ammonia, alkanolamines and/or basic amino acids.

Preferably, the alkanolamine(s) are chosen from monoethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethylamino)methane.

More preferentially, the alkanolamine(s) are chosen from monoethanolamine and/or 2-amino-2-methyl-1-propanol.

For the purposes of the present invention, the term “amino acid” means organic compounds containing two functional groups: both a carboxyl —COOH or carboxylate group and an amine group —NH₂, it being possible for the amine group to be optionally methylated, i.e. in the form —NR₂ or N⁺R₃, where at least one R═CH₃.

Preferably, the amino acid(s) are chosen from aminocarboxylic acids such as alpha-aminocarboxylic acid.

The term “basic amino acid” is intended to mean amino acids having an isoelectric point above 7.

Preferably, the basic amino acid(s) are chosen from arginine, lysine, ornithine and/or histidine, more preferentially arginine and/or lysine.

According to one particular embodiment, composition A and/or composition B can comprise an inorganic alkaline agent, preferably chosen from ammonium hydroxide, sodium hydroxide, potassium hydroxide, calcium hydroxide, barium hydroxide, sodium phosphate, potassium phosphate, sodium silicate, sodium meta-silicate, potassium silicate, sodium carbonate and/or potassium carbonate.

Composition A and/or composition B may also comprise acidifying agents to adjust the pH.

Preferably, the acidifying agents are chosen from citric acid, lactic acid, acetic acid and/or mineral acids such as hydrochloric acid, sulfuric acid, phosphoric acid and mixtures thereof.

### Additives:

Composition A and/or composition B according to the invention may also contain any commonly used adjuvant or additive.

Among the additives that may be contained in the composition, mention may be made of reducing agents, thickeners other than the polymers described previously, softeners, antifoams, moisturizers, UV-screening agents, peptizers, dispersants, solubilizers, fragrances, anionic, cationic, non-ionic or amphoteric surfactants, proteins, vitamins, preserving agents, fillers, oils, waxes and mixtures thereof.

Composition A and/or composition B may be applied notably on the hair in the form of a spray, a cream, a gel or a foam.

According to a particular embodiment, composition A according to the invention is an anhydrous liquid or an anhydrous gel.

According to a particular embodiment, composition B according to the invention is a dispersion.

According to one preferred embodiment, composition A according to the invention comprises at least one alkoxysilane of formula (I) as described previously, at least one alkoxysilane of formula (II) as described previously, and at least one pigment.

According to a more preferred embodiment, composition A according to the invention comprises 3-aminopropyltriethoxysilane (APTES), methyltrimethoxysilane (MTMS) or methyltriethoxysilane (MTES) and at least one pigment.

According to one preferred embodiment, composition B according to the invention comprises at least one film-forming polymer chosen from vinylpyrrolidone homopolymers (PVP), vinylpyrrolidone copolymers, acrylamide copolymers, acrylic acid ester homopolymers or copolymers, ethylene homopolymers or copolymers and mixtures thereof.

According to a more preferred embodiment, composition B according to the invention comprises at least one film-forming polymer chosen from vinylpyrrolidone homopolymers (PVP), acrylamide copolymers, acrylic acid ester homopolymers or copolymers, ethylene homopolymers or copolymers and mixtures thereof.

### Spray Device

As indicated previously, compositions A and/or B are packaged in one or more spray devices.

According to one preferred embodiment, composition A as described previously is packaged in a spray device.

According to another preferred embodiment, composition B as described previously is packaged in a spray device.

According to another preferred embodiment, composition A as described previously is packaged in a first spray device, and composition B as described previously is packaged in a second spray device.

According to another preferred embodiment, compositions A and B are packaged in the same spray device.

Preferably, the spray device according to the invention comprises a container containing composition A and/or composition B equipped with a spray mechanism for delivering composition A and/or composition B in the form of droplets.

The walls of the container are rigid or non-rigid, preferably rigid, the container possibly being in this case a can, for example made of metal or plastic.

The spray device may also comprise a pushbutton which allows the user, by pressing on top, to trigger the spraying of composition A and/or composition B according to the invention on the keratin fibres, such as the hair.

The spray device may be equipped with a spray mechanism comprising a dispensing pump.

The dispensing pump may comprise a tubular body corresponding to the means for withdrawing composition A and/or composition B inside the container.

The dispensing pump may be mounted on the container comprising composition A and/or composition B.

The dispensing pump is preferably a manually actuated pump. Thus, during the manual displacement of the pushbutton, the dispensing pump is actuated in order to supply the tubular body of the pump with composition A and/or composition B according to the invention.

The spray mechanism may also comprise a spray nozzle. This spray nozzle is mounted so as to form a path for dispensing composition A and/or composition B according to the invention between the dispensing pump and a dispensing orifice.

Preferably, the spray nozzle is mounted in the pushbutton.

According to a preferred embodiment, the device for spraying composition A and/or composition B comprises at least one container containing composition A and/or composition B equipped with a spray mechanism provided with at least one dispensing orifice, in communication with the container.

In order to use the spray device, the user presses on the pushbutton, triggering a spraying of composition A and/or composition B according to the invention on the keratin fibres, such as the hair.

Such spray devices are described in the patent application FR2952360A1 filed by Rexam Dispensing Systems or the patent application FR2718372A1 filed by the company Sofab.

The spray device is not limited to the implementational examples which have just been described.

The present invention thus relates to a device for spraying composition A and/or composition B comprising at least one container containing composition A and/or composition B as described previously equipped with a spray mechanism provided with at least one dispensing orifice, in communication with the container.

According to one preferred embodiment, the spray device according to the invention comprises:
at least one container containing composition A comprising:
(i) at least one alkoxysilane chosen from the compounds of formula (I) or of formula (I′) as described previously, oligomers thereof and/or mixtures thereof, and
(ii) at least one alkoxysilane of formula (II) as described previously, oligomers thereof and/or mixtures thereof, and
(iii) at least one colouring agent chosen from pigments, direct dyes and mixtures thereof, and
a spray mechanism provided with at least one dispensing orifice, in communication with the container.

Advantageously, the spray device according to the invention comprises:
at least one container containing composition A comprising:
(i) at least one alkoxysilane of formula (I) as described previously, oligomers thereof and/or mixtures thereof, and
(ii) at least one alkoxysilane of formula (II) as described previously, oligomers thereof and/or mixtures thereof, and
(iii) at least one pigment,
a spray mechanism provided with at least one dispensing orifice, in communication with the container.

According to one preferred embodiment, the spray device according to the invention comprises:
at least one container containing composition B comprising:
(i) at least one film-forming polymer as described previously,
(ii) optionally, at least one colouring agent chosen from pigments, direct dyes and mixtures thereof, and
a spray mechanism provided with at least one dispensing orifice, in communication with the container.

According to one preferred embodiment, the spray device according to the invention comprises:
at least one container containing composition B comprising at least one film-forming polymer chosen from vinylpyrrolidone homopolymers (PVP), vinylpyrrolidone copolymers, acrylamide copolymers, acrylic acid ester homopolymers or copolymers, ethylene homopolymers or copolymers and mixtures thereof, and
a spray mechanism provided with at least one dispensing orifice, in communication with the container.

According to one preferred embodiment, compositions A and/or B are packaged in one or more spray devices in the presence of at least one propellant as defined below.

Advantageously, said propellant is chosen from optionally halogenated volatile hydrocarbons, in particular alkanes, such as n-butane, propane, isobutane, pentane and halogenated derivatives thereof; carbon dioxide, nitrous oxide, dimethyl ether, nitrogen, compressed air, alone or as mixtures.

Preferably, said propellant is chosen from alkanes, even more preferentially from n-butane, propane, isobutane, and mixtures thereof.

The propellant(s) are pressurized, more particularly at least partially in liquid form.

Advantageously, the total amount of propellant(s) ranges from 1 to 99% by weight, preferably from 1 to 98% by weight, more preferentially from 2 to 95% by weight, relative to the weight of the composition containing it (them).

According to one preferred embodiment, composition A as described previously is packaged in a spray device in the presence of at least one propellant.

According to another preferred embodiment, composition B as described previously is packaged in a spray device in the presence of at least one propellant.

According to another preferred embodiment, composition A as described previously is packaged in a first spray device in the presence of at least one propellant, and composition B as described previously is packaged in a second spray device in the presence of at least one propellant.

According to another preferred embodiment, composition A as described previously and composition B as described previously are packaged in the same spray device in the presence of at least one propellant. According to this embodiment, compositions A and B are preferably in separate compartments.

According to one preferred embodiment, the spray device according to the invention comprises:
at least one container containing at least one propellant as defined previously and composition A comprising:
(i) at least one alkoxysilane chosen from the compounds of formula (I) or of formula (I′) as described previously, oligomers thereof and/or mixtures thereof, and
(ii) at least one alkoxysilane of formula (II) as described previously, oligomers thereof and/or mixtures thereof, and
(iii) at least one colouring agent chosen from pigments, direct dyes and mixtures thereof, and
a spray mechanism provided with at least one dispensing orifice, in communication with the container.

According to one preferred embodiment, the spray device according to the invention comprises:
at least one container containing at least one propellant as defined previously and composition B comprising:
(i) at least one film-forming polymer as described previously,
(ii) optionally, at least one colouring agent chosen from pigments, direct dyes and mixtures thereof, and
a spray mechanism provided with at least one dispensing orifice, in communication with the container.

### Process for Treating Keratin Fibres

A subject of the present invention is a process for dyeing keratin fibres such as the hair, comprising the following steps:
a) applying to the keratin fibres at least one composition A comprising:
at least one alkoxysilane chosen from the compounds of formula (I) or of formula (I′) as described previously, oligomers thereof and/or mixtures thereof, and
at least one alkoxysilane of formula (II) as described previously, oligomers thereof and/or mixtures thereof, and
b) applying to the keratin fibres at least one composition B comprising at least one film-forming polymer as described previously, composition A and/or composition B comprising at least one colouring agent chosen from pigments, direct dyes and mixtures thereof, and composition A and/or composition B being packaged in one or more spray devices as described previously.

Preferably, composition A and composition B according to the invention are compositions for dyeing keratin fibres such as the hair.

Composition A and composition B can be applied simultaneously or sequentially. Preferably, composition A and composition B are applied sequentially.

Thus, preferably, composition A is first of all applied to the keratin fibres, followed by composition B in a second step.

According to a preferred embodiment, the process for dyeing keratin fibres such as the hair comprises:
i) applying to said keratin fibres composition A according to the invention comprising:
at least one alkoxysilane chosen from the compounds of formula (I) or of formula (I′) as described previously, oligomers thereof and/or mixtures thereof,
at least one alkoxysilane of formula (II) as described previously, oligomers thereof and/or mixtures thereof, and
at least one colouring agent chosen from pigments, direct dyes and mixtures thereof,
ii) optionally a leave-on time of from 10 seconds to 20 minutes, notably from 20 seconds to 10 minutes, preferably from 30 seconds to 5 minutes, of composition A on said fibres,
iii) optionally a step of washing, rinsing, wringing out or drying said fibres,
iv) applying to said keratin fibres composition B according to the invention, packaged in a spray device according to the invention, comprising at least one film-forming polymer as described previously, and
v) optionally a leave-on time of from 10 seconds to 20 minutes, notably from 20 seconds to 10 minutes, preferably from 30 seconds to 5 minutes, of composition B according to the invention on said fibres,
vi) optionally a step of washing, rinsing, wringing out and
vii) optionally a step of drying and/or heating the keratin fibres for example at a temperature of greater than or equal to 30° C.

According to a preferred embodiment, the process for dyeing keratin fibres such as the hair comprises:
i) applying to said keratin fibres composition A according to the invention, packaged in a spray device according to the invention, comprising:
at least one alkoxysilane chosen from the compounds of formula (I) or of formula (I′) as described previously, oligomers thereof and/or mixtures thereof,
at least one alkoxysilane of formula (II) as described previously, oligomers thereof and/or mixtures thereof, and
at least one colouring agent chosen from pigments, direct dyes and mixtures thereof,
ii) optionally a leave-on time of from 10 seconds to 20 minutes, notably from 20 seconds to 10 minutes, preferably from 30 seconds to 5 minutes, of composition A on said fibres,
iii) optionally a step of washing, rinsing, wringing out or drying said fibres,
iv) applying to said keratin fibres composition B according to the invention, comprising at least one film-forming polymer as described previously, and
v) optionally a leave-on time of from 10 seconds to 20 minutes, notably from 20 seconds to 10 minutes, preferably from 30 seconds to 5 minutes, of composition B according to the invention on said fibres,
vi) optionally a step of washing, rinsing, wringing out and
vii) optionally a step of drying and/or heating the keratin fibres for example at a temperature of greater than or equal to 30° C.

According to a preferred embodiment, the process for dyeing keratin fibres such as the hair comprises:
i) applying to said keratin fibres composition A according to the invention, packaged in a spray device according to the invention, comprising:
at least one alkoxysilane chosen from the compounds of formula (I) or of formula (I′) as described previously, oligomers thereof and/or mixtures thereof,
at least one alkoxysilane of formula (II) as described previously, oligomers thereof and/or mixtures thereof, and
at least one colouring agent chosen from pigments, direct dyes and mixtures thereof,
ii) optionally a leave-on time of from 10 seconds to 20 minutes, notably from 20 seconds to 10 minutes, preferably from 30 seconds to 5 minutes, of composition A on said fibres,
iii) optionally a step of washing, rinsing, wringing out or drying said fibres,
iv) applying to said keratin fibres composition B according to the invention, packaged in a spray device according to the invention, comprising at least one film-forming polymer as described previously, and
v) optionally a leave-on time of from 10 seconds to 20 minutes, notably from 20 seconds to 10 minutes, preferably from 30 seconds to 5 minutes, of composition B according to the invention on said fibres,
vi) optionally a step of washing, rinsing, wringing out and
vii) optionally a step of drying and/or heating the keratin fibres for example at a temperature of greater than or equal to 30° C.

According to a preferred embodiment, the process for dyeing keratin fibres such as the hair comprises:
i) applying to said keratin fibres composition A according to the invention comprising:
at least one alkoxysilane chosen from the compounds of formula (I) or of formula (I′) as described previously, oligomers thereof and/or mixtures thereof,
at least one alkoxysilane of formula (II) as described previously, oligomers thereof and/or mixtures thereof, and
at least one colouring agent chosen from pigments, direct dyes and mixtures thereof,
ii) optionally a leave-on time of from 10 seconds to 20 minutes, notably from 20 seconds to 10 minutes, preferably from 30 seconds to 5 minutes, of composition A on said fibres,
iii) optionally a step of washing, rinsing, wringing out or drying said fibres,
iv) applying to said keratin fibres composition B according to the invention, packaged in a spray device according to the invention, comprising:
at least one film-forming polymer as described previously, and
optionally at least one colouring agent chosen from pigments, direct dyes and mixtures thereof,
v) optionally a leave-on time of from 10 seconds to 20 minutes, notably from 20 seconds to 10 minutes, preferably from 30 seconds to 5 minutes, of composition B according to the invention on said fibres,
vi) optionally a step of washing, rinsing, wringing out and
vii) optionally a step of drying and/or heating the keratin fibres for example at a temperature of greater than or equal to 30° C.

A “rinsing step” is understood to mean the application of water to the keratin fibres.

Compositions A and B according to the invention can be used on wet or dry keratin fibres, and also on all types of fair or dark, natural or dyed, permanent-waved, bleached or relaxed, fibres.

According to one particular embodiment of the process of the invention, the fibres are washed before applying compositions A and B according to the invention.

If the application of composition A and/or of composition B according to the invention to the keratin fibres is not carried out using the spray device according to the invention, it can be carried out by any conventional means, in particular using a comb, a fine brush, a coarse brush or with the fingers.

The dyeing process, i.e. application of compositions A and B according to the invention to the keratin fibres, is generally carried out at room temperature (between 15° C. and 25° C.).

After application of composition A or composition B to the keratin fibres, it is preferable to have a leave-on time of from 10 seconds to 20 minutes, notably from 20 seconds to 10 minutes, preferably from 30 seconds to 5 minutes, of composition A or composition B on said fibres.

According to a preferred embodiment, the process for dyeing keratin fibres such as the hair comprises:
i) preparing composition A according to the invention by mixing a first composition A′ and a second composition A″ in which the first composition A′ comprises:
at least one alkoxysilane chosen from the compounds of formula (I) or of formula (I′) as described previously, oligomers thereof and/or mixtures thereof, and
at least one alkoxysilane of formula (II) as described previously, oligomers thereof and/or mixtures thereof, and
the second composition A″ comprises:
at least one colouring agent chosen from pigments, direct dyes and mixtures thereof,
ii) applying to said keratin fibres composition A according to the invention,
iii) optionally a leave-on time of from 10 seconds to 20 minutes, notably from 20 seconds to 10 minutes, preferably from 30 seconds to 5 minutes, of composition A on said fibres,
iv) optionally a step of washing, rinsing, wringing out or drying said fibres,
v) applying composition B according to the invention, packaged in a spray device according to the invention, comprising at least one film-forming polymer as described previously, and
vi) optionally a leave-on time of from 10 seconds to 20 minutes, notably from 20 seconds to 10 minutes, preferably from 30 seconds to 5 minutes, of composition B according to the invention on said fibres,
vii) optionally a step of washing, rinsing, wringing out and
viii) optionally a step of drying and/or heating the keratin fibres for example at a temperature of greater than or equal to 30° C.

According to a particular embodiment, the process for dyeing keratin fibres such as the hair comprises:
i) preparing composition A according to the invention by mixing a first composition A′ packaged in a spray device according to the invention, and a second composition A″ packaged in a spray device according to the invention, in which the first composition A′ comprises:
at least one alkoxysilane chosen from the compounds of formula (I) or of formula (I′) as described previously, oligomers thereof and/or mixtures thereof, and
at least one alkoxysilane of formula (II) as described previously, oligomers thereof and/or mixtures thereof, and
the second composition A″ comprises:
at least one colouring agent chosen from pigments, direct dyes and mixtures thereof,
ii) applying to said keratin fibres composition A according to the invention,
iii) optionally a leave-on time of from 10 seconds to 20 minutes, notably from 20 seconds to 10 minutes, preferably from 30 seconds to 5 minutes, of composition A on said fibres,
iv) optionally a step of washing, rinsing, wringing out or drying said fibres,
v) applying composition B according to the invention, packaged in a spray device according to the invention, comprising at least one film-forming polymer as described previously, and
vi) optionally a leave-on time of from 10 seconds to 20 minutes, notably from 20 seconds to 10 minutes, preferably from 30 seconds to 5 minutes, of composition B according to the invention on said fibres,
vii) optionally a step of washing, rinsing, wringing out and
viii) optionally a step of drying and/or heating the keratin fibres for example at a temperature of greater than or equal to 30° C.

According to another particular embodiment, the process for dyeing keratin fibres such as the hair comprises:
i) the use of a spray device comprising at least two compartments a and b separate from one another, compartment a comprising a composition A′ comprising:
at least one alkoxysilane chosen from the compounds of formula (I) or of formula (I′) as described previously, oligomers thereof and/or mixtures thereof, and
at least one alkoxysilane of formula (II) as described previously, oligomers thereof and/or mixtures thereof,
compartment b containing a composition A″ comprising at least one colouring agent chosen from pigments, direct dyes and mixtures thereof,
said spray device comprising a spray mechanism provided with at least one dispensing orifice, in communication with compartments a and b, for simultaneously delivering compositions A′ and A″ in order to obtain composition A according to the invention,
ii) applying to said keratin fibres composition A according to the invention,
iii) optionally a leave-on time of from 10 seconds to 20 minutes, notably from 20 seconds to 10 minutes, preferably from 30 seconds to 5 minutes, of composition A on said fibres,
iv) optionally a step of washing, rinsing, wringing out or drying said fibres,
v) applying composition B according to the invention, comprising at least one film-forming polymer as described previously, and
vi) optionally a leave-on time of from 10 seconds to 20 minutes, notably from 20 seconds to 10 minutes, preferably from 30 seconds to 5 minutes, of composition B according to the invention on said fibres,
vii) optionally a step of washing, rinsing, wringing out and
viii) optionally a step of drying and/or heating the keratin fibres for example at a temperature of greater than or equal to 30° C.

According to another particular embodiment, the process for dyeing keratin fibres such as the hair comprises:
i) the use of a spray device comprising at least two compartments a and b separate from one another,
compartment a comprising a composition A′ comprising:
at least one alkoxysilane chosen from the compounds of formula (I) or of formula (I′) as described previously, oligomers thereof and/or mixtures thereof, and
at least one alkoxysilane of formula (II) as described previously, oligomers thereof and/or mixtures thereof,
compartment b containing a composition A″ comprising at least one colouring agent chosen from pigments, direct dyes and mixtures thereof,
said spray device comprising a spray mechanism provided with at least one dispensing orifice, in communication with compartments a and b, for simultaneously delivering compositions A′ and A″ in order to obtain composition A according to the invention,
ii) applying to said keratin fibres composition A according to the invention,
iii) optionally a leave-on time of from 10 seconds to 20 minutes, notably from 20 seconds to 10 minutes, preferably from 30 seconds to 5 minutes, of composition A on said fibres,
iv) optionally a step of washing, rinsing, wringing out or drying said fibres,
v) applying composition B according to the invention, packaged in a spray device according to the invention, comprising at least one film-forming polymer as described previously, and
vi) optionally a leave-on time of from 10 seconds to 20 minutes, notably from 20 seconds to 10 minutes, preferably from 30 seconds to 5 minutes, of composition B according to the invention on said fibres,
vii) optionally a step of washing, rinsing, wringing out and
viii) optionally a step of drying and/or heating the keratin fibres for example at a temperature of greater than or equal to 30° C.

In order to allow better preservation of composition A′, the latter preferably comprises a low water content or is anhydrous.

Preferably, composition A′ comprises a water content of between 0.001 and 10% by weight, better still between 0.5 and 10% by weight, more preferentially between 1 and 8% by weight relative to the weight of composition A′.

Composition A″ may also contain water.

After applying compositions A and B according to the invention and before a possible step of applying heat to the keratin fibres, the keratin fibres may be subjected to a drying step, for example using a hairdryer.

The drying step may be performed for a period of between 20 seconds and 5 minutes.

The drying step may be performed using absorbent paper, a hairdryer or a styling hood or by drying naturally.

Preferably, the drying step is performed using a hairdryer at a temperature greater than or equal to 30° C., more particularly at a temperature above 30° C. and below 110° C.

After applying compositions A and B according to the invention to the keratin fibres, it is possible to wait at least 10 seconds, preferably at least 30 seconds, before the step of drying the keratin fibres.

After application of compositions A and B according to the invention and optionally a step of drying the keratin fibres, the process according to the invention may comprise a step of applying heat to the keratin fibres using a heating tool.

Preferably, the process according to the invention does not comprise a step of applying heat to the keratin fibres using a heating tool in addition to the possible drying step.

The heat application step of the process of the invention can be carried out using a hood, a hairdryer, a straightening iron, a curling iron, a Climazon.

Preferably, the heat application step of the process of the invention is performed using a hairdryer and/or a straightening iron, more preferentially using a hairdryer.

During the step of applying heat to the keratin fibres, a mechanical action may be exerted on the locks, such as combing, brushing or running the fingers through.

When the step of applying heat to the keratin fibres is performed using a hood or a hairdryer, the temperature is preferably between 30° C. and 110° C., preferentially between 50° C. and 90° C.

When the step of applying heat to the keratin fibres is performed using a straightening iron, the temperature is preferably between 110° C. and 240° C., preferably between 110° C. and 200° C.

### Multicompartment Device (Kit)

The present invention also relates to a kit for dyeing keratin fibres such as the hair, comprising several compartments containing:
in a first compartment, a composition A comprising:
at least one alkoxysilane chosen from the compounds of formula (I) or of formula (I′) as described previously, oligomers thereof and/or mixtures thereof,
at least one alkoxysilane of formula (II) as described previously, oligomers thereof and/or mixtures thereof,
in a second compartment, a composition B according to the invention comprising at least one film-forming polymer as described previously, composition A and/or composition B comprising at least one colouring agent chosen from pigments, direct dyes and mixtures thereof, and
composition A and/or composition B being packaged in one or more spray devices.

According to a preferred embodiment, the dyeing kit according to the invention is a kit for dyeing keratin fibres such as the hair, comprising several compartments containing:
in a first compartment, a composition A comprising:
at least one alkoxysilane chosen from the compounds of formula (I) or of formula (I′) as described previously, oligomers thereof and/or mixtures thereof,
at least one alkoxysilane of formula (II) as described previously, oligomers thereof and/or mixtures thereof, and
at least one colouring agent chosen from pigments, direct dyes and mixtures thereof,
in a second compartment, a composition B according to the invention comprising at least one film-forming polymer as described previously, composition A and/or composition B being packaged in one or more spray devices.

The present invention also relates to a kit for dyeing keratin fibres such as the hair, comprising several compartments containing:
in a first compartment, a composition A′ comprising:
at least one alkoxysilane chosen from the compounds of formula (I) or of formula (I′) as described previously, oligomers thereof and/or mixtures thereof,
at least one alkoxysilane of formula (II) as described previously, oligomers thereof and/or mixtures thereof,
in a second compartment, a composition A″ comprising at least one colouring agent chosen from pigments, direct dyes and mixtures thereof,
in a third compartment, a composition B according to the invention comprising at least one film-forming polymer as described previously,
composition A′ and/or composition A″ and/or composition B being packaged in one or more spray devices.

The present invention will now be described more specifically by means of examples, which do not in any way limit the scope of the invention. However, the examples make it possible to support specific features, variants and preferred embodiments of the invention.

### EXAMPLE

In the examples, the temperature is given in degrees Celsius and corresponds to room temperature (20-25° C.), unless otherwise indicated, and the pressure is atmospheric pressure, unless otherwise indicated.

The following composition is prepared (in g/100 g of starting material as obtained):

TABLE 1

| Composition | A′ |
|---|---|
| 3-Aminopropyltriethoxysilane | 24 |
| (APTES) ⁽¹⁾ | |
| Methyltriethoxysilane (MTES) | 72 |
| Ethanol | q.s. |
| | for 100 |

TABLE 2

| Composition | A″ |
|---|---|
| Pigment⁽²⁾ | 10 |
| Hydroxypropyl | 4 |
| cellulose | |
| Ethanol | q.s. |
| | for 100 |

TABLE 3

| Composition | B |
|---|---|
| Ethylene/sodium | 15 |
| acrylate copolymer ⁽³⁾ | |
| Water | q.s. |
| | for 100 |

(1) Sold under the trade name KBE-903 by the company Shin-Etsu;
(2) Synthetic Fluorphlogopite (and) Titanium Dioxide (and) Tin Oxide sold under the trade name Syncrystal Silk Silver by the company Eckart;
(3) Sold under the trade name Ecosmooth Satin by the company Dow Corning (containing 25% active material in water).

Two different processes were applied to the locks of hair:
a comparative hair dyeing process in which composition B is applied with the fingers to the locks of hair,
a hair dyeing process according to the invention in which composition B is packaged in a spray device and applied to the locks of hair using a spray device. The spray device comprises a conventional bottle and a pump (commercial reference PZ20; brand Costair) which delivers 290 μl for each press.

### Protocol:

Compositions A′ and A″ were mixed in a 50/50 ratio by weight in order to obtain composition A.
According to the comparative dyeing process, composition A was applied by finger to locks of dry chestnut-brown hair (TD4), in a proportion of 1 g of composition per gram of lock.

The locks of hair were left for 2 minutes at room temperature and then dried with a hairdryer.

Composition B was then applied by finger to the locks of hair treated with composition A, in a proportion of 1 g of composition per gram of lock.

The locks of hair were left for 5 minutes at room temperature.

The locks of hair were then dried with a hairdryer for 2 minutes.

The locks of hair were left at room temperature for 24 hours.
According to the dyeing process according to the invention, composition A was applied by finger to locks of dry chestnut-brown hair (TD4), in a proportion of 1 g of composition per gram of lock.

The locks of hair were left for 2 minutes at room temperature and then dried with a hairdryer.

Composition B according to the invention was packaged in the spray device as described previously.

Composition B according to the invention was then applied using this spray device to the locks of hair treated with composition A suspended vertically, in a proportion of 1 g of composition per gram of lock.

The locks of hair thus treated were left for 5 minutes at room temperature and then dried with a hairdryer for 2 minutes.

The locks of hair were left at room temperature for 24 hours.

The locks of hair thus dyed were subjected to colorimetric measurements so as to evaluate the coverage of the initial hair colour by the coloured coating and also the visibility of the coloured coating.

Additionally, the locks of hair thus dyed were subjected to a shampoo wash so as to evaluate the fastness (persistence) of the colouring obtained with respect to shampoo washing, according to the shampoo washing protocol described below.

### Shampoo Washing Protocol:

The following shampoo washing operation was carried out on the locks obtained after the application of the hairdryer.

The locks of hair were washed with a standard shampoo (Gamier Ultra Doux).

The locks of hair were then rinsed, combed and dried with a hairdryer.

### Results

The persistence of the colour of the locks with respect to shampoo washing and also the modification of the initial colour were evaluated in the CIE L*a*b* system, using a Minolta Spectrophotometer CM3600A colorimeter (illuminant D65, angle 100, specular component included).

In this L*a*b* system, L* represents the intensity of the colour, a* indicates the green/red colour axis and b* the blue/yellow colour axis.

The persistence of the colouring is evaluated by the colour difference ΔE between the coloured locks before shampooing, then after having undergone one shampoo wash according to the protocol described above. The lower the ΔE value, the more persistent the colour with respect to shampoo washing.

The modification of the colour is evaluated by the colour difference ΔE between the control (undyed) locks and the locks dyed according to the processes described above. The higher the ΔE value, the greater the modification of the initial colour.

The ΔE value is calculated according to the following equation:

$$Δ E = \sqrt{{( {L}^{*} - {L}_{o}^{*} )}^{2} + {( {a}^{*} - {a}_{o}^{*} )}^{2} + {( {b}^{*} - {b}_{o}^{*} )}^{2}} \qquad [ Math \text{.1} ]$$

In this equation, for the persistence of the colouring with respect to shampoo washing, L*, a* and b* represent the values measured after dyeing the hair and after having undergone a shampoo wash, and L0*, a0* and b0* represent the values measured after dyeing to hair but before shampoo washing.

For the modification of the colour, L*, a* and b* represent the values measured after dyeing the hair, and L0*, a0* and b0* represent the values measured before dyeing the hair.

The results are shown in the table below:

TABLE 4

| | | | | | | ΔE with |
|---|---|---|---|---|---|---|
| | | | | | ΔE with | respect to the |
| | | | | | respect to | lock before |
| | Number of | | | | the | and after |
| | shampoo | | | | control | shampoo |
| Method | washes | L* | a* | b* | lock | washing |
| Control lock | — | 21.4 | 3.5 | 3.9 | — | — |
| Process | 0 | 41.5 | 1.4 | −1.1 | 20.8 | — |
| according to | 1 | 39.5 | 1.6 | −1.3 | 18.9 | 2.0 |
| the invention | | | | | | |
| Comparative | 0 | 37.2 | 1.3 | −1.3 | 16.8 | — |
| process | 1 | 30.7 | 1.8 | 0.2 | 10.1 | 6.7 |

The dyeing process according to the invention results in higher values of ΔE with respect to the control lock than the comparative dyeing process.

Specifically, the use of the dyeing process according to the invention results in a colouring that is more visible and that exhibits good coverage of the initial hair colour.

Furthermore, the locks of hair dyed with the dyeing process according to the invention and washed with a shampoo have low values of ΔE with respect to the locks of hair dyed with the comparative dyeing process and washed with a shampoo.

Specifically, the locks of hair dyed with the dyeing process according to the invention and washed with a shampoo exhibit better persistence of the colour.

Thus, the coloured coating of the keratin fibres obtained with the dyeing process according to the invention exhibits good persistence with respect to shampoo washes.

1. Process for dyeing human keratin fibres such as the hair, comprising the following steps:
a) applying to the keratin fibres at least one composition A comprising:
at least one alkoxysilane chosen from the compounds of formula (I) or formula (I′) below, oligomers thereof and/or mixtures thereof: 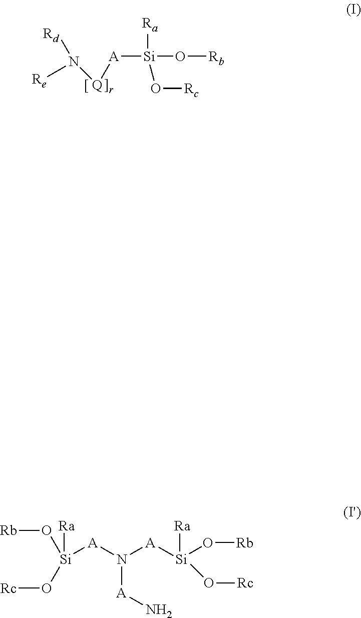
in which:
Rₐ represents an alkyl group containing from 1 to 20 carbon atoms, preferably from 1 to 10 carbon atoms, more preferentially from 1 to 4 carbon atoms and in particular from 1 to 2 carbon atoms such as a methyl, said alkyl group being optionally substituted with an aryl group; an alkoxy group containing from 1 to 10 carbon atoms, preferably from 1 to 4 carbon atoms and in particular from 1 to 2 carbon atoms such as an ethoxy; or an aryl group containing from 6 to 12 carbon atoms;
R_{b} and R_{c}, which may be identical or different, represent a hydrogen atom; an alkyl group containing from 1 to 20 carbon atoms, preferably from 1 to 6 carbon atoms and notably from 1 to 4 carbon atoms, notably an ethyl group, it being understood that if Rₐ does not represent an alkoxy group, then R_{b} and R_{c} cannot simultaneously represent a hydrogen atom;
R_{d} and Rₑ, which may be identical or different, represent a hydrogen atom; an alkyl group containing from 1 to 20 carbon atoms, preferably from 1 to 6 carbon atoms and notably from 1 to 4 carbon atoms; a cycloalkyl group containing from 3 to 20 carbon atoms; an aryl group containing from 6 to 12 carbon atoms; an aminoalkyl group containing from 1 to 20 carbon atoms;
A independently represents a linear or branched alkylene group containing from 1 to 10 carbon atoms, which may be interrupted with at least one heteroatom chosen from O, S, NH or a carbonyl group (CO), preferably NH;
Q represents a carbonyl group (CO);
r denotes an integer ranging from 0 to 1; and
at least one alkoxysilane of formula (II) below, oligomers thereof and/or mixtures thereof: 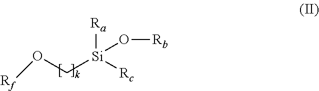
in which:
Rₐ represents an alkyl group containing from 1 to 20 carbon atoms, preferably from 1 to 10 carbon atoms, more preferentially from 1 to 4 carbon atoms and in particular from 1 to 2 carbon atoms such as a methyl, said alkyl group being optionally substituted with an aryl group; an alkoxy group containing from 1 to 10 carbon atoms, preferably from 1 to 4 carbon atoms and in particular from 1 to 2 carbon atoms such as an ethoxy; or an aryl group containing from 6 to 12 carbon atoms;
R_{b} represents a hydrogen atom or an alkyl group containing from 1 to 20 carbon atoms, preferably from 1 to 6 carbon atoms and notably from 1 to 4 carbon atoms, notably an ethyl group;
R_{c} represents an alkyl group containing from 1 to 20 carbon atoms, preferably from 1 to 10 carbon atoms, more preferentially from 1 to 4 carbon atoms and in particular from 1 to 2 carbon atoms such as a methyl, said alkyl group being optionally substituted with an aryl group; an alkoxy group containing from 1 to 10 carbon atoms, preferably from 1 to 4 carbon atoms and in particular from 1 to 2 carbon atoms such as an ethoxy; or an aryl group containing from 6 to 12 carbon atoms; it being understood that, if Rₐ and Rₑ do not represent an alkoxy group, then R_{b} cannot represent a hydrogen atom;
k denotes an integer ranging from 0 to 5, preferably ranging from 0 to 3;
R_{f} represents a hydrogen atom; an alkyl group containing from 1 to 10 carbon atoms and notably from 1 to 4 carbon atoms; or a group of formula (IIa) below: 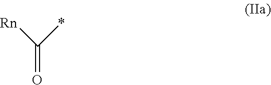
in which Rₙ represents a hydroxyl group (OH); an alkyl group containing from 1 to 10 carbon atoms, preferably a methyl, and
b) applying to the keratin fibres at least one composition B comprising at least one film-forming polymer,
composition A and/or composition B comprising at least one colouring agent chosen from pigments, direct dyes and mixtures thereof, and
composition A and/or composition B being packaged in one or more spray devices.

2. Process according to claim 1, in which the alkoxysilane(s), oligomers thereof and/or mixtures thereof are chosen from the compounds of formula (I) in which:
Rₐ represents an alkyl group containing from 1 to 10 carbon atoms, notably from 1 to 4 carbon atoms and in particular from 1 to 2 carbon atoms, preferably a methyl, or an alkoxy group containing from 1 to 4 carbon atoms, preferably from 1 to 2 carbon atoms, preferably an ethoxy;
R_{b} and R_{c}, which may be identical or different, represent an alkyl group containing from 1 to 10 carbon atoms, preferably from 1 to 4 carbon atoms, such as an ethyl;
R_{d} and Rₑ, which are identical, represent a hydrogen atom or R_{d} denotes a hydrogen atom and Rₑ denotes a C₅-C₆ cycloalkyl radical such as cyclohexyl;
A independently represents a linear or branched alkylene group containing from 1 to 10 carbon atoms, which may be interrupted with at least one heteroatom chosen from O, S and NH or a carbonyl group (CO), preferably NH;
r denotes an integer equal to 0.

3. Process according to claim 1, in which the alkoxysilane(s), oligomers thereof and/or mixtures thereof are chosen from the compounds of formula (I) in which Rₐ represents an ethoxy group, R_{b} and R_{c} are identical and represent an ethyl, R_{d} and Rₑ represent a hydrogen atom, A represents a propylene and r denotes an integer equal to 0.

4. Process according to claim 1, in which the alkoxysilane(s) of formula (I) or of formula (I′), oligomers thereof and/or mixtures thereof is (are) present in a total amount ranging from 0.1 to 40% by weight, preferably from 0.5 to 30% by weight, preferentially from 0.75 to 25% by weight, better still from 1 to 20% by weight and even better still from 1.5 to 15% by weight, relative to the total weight of composition A.

5. Process according to claim 1, in which the alkoxysilane(s) of formula (II), oligomers thereof and/or mixtures thereof, are such that:
Rₐ represents an alkyl group containing from 1 to 10 carbon atoms, preferably from 1 to 4 carbon atoms, in particular from 1 to 2 carbon atoms, such as a methyl or an ethyl;
R_{b} represents an alkyl group containing from 1 to 10 carbon atoms, preferably from 1 to 4 carbon atoms, in particular from 1 to 2 carbon atoms, such as a methyl or an ethyl;
R_{c} represents an alkoxy group containing from 1 to 10 carbon atoms, preferably from 1 to 4 carbon atoms and in particular from 1 to 2 carbon atoms, such as a methoxy or an ethoxy;
k denotes an integer equal to 0;
R_{f} represents an alkyl group containing from 1 to 10 carbon atoms and notably from 1 to 4 carbon atoms, such as a methyl or an ethyl,
preferably methyltrimethoxysilane (MTMS) or methyltriethoxysilane (MTES).

6. Process according to claim 1, in which the alkoxysilane(s) of formula (II), oligomers thereof and/or mixtures thereof is (are) present in a total amount ranging from 0.5 to 90% by weight, preferably from 1 to 75% by weight, preferentially from 3 to 45% by weight and better still from 5 to 40% by weight relative to the total weight of composition A.

7. Process according to claim 1, in which the film-forming polymer(s) are chosen from vinylpyrrolidone homopolymers (PVP), acrylamide copolymers, acrylic acid ester homopolymers or copolymers, ethylene homopolymers or copolymers and mixtures thereof.

8. Process according to claim 1, in which the film-forming polymer(s) is (are) present in a total amount ranging from 0.1 to 30% by weight, preferably from 0.5 to 25% by weight and better still from 1 to 20% by weight, relative to the total weight of composition B.

9. Process according to claim 1, in which composition A and/or composition B comprise(s) at least one non-associative, non-ionic cellulose-based polymer chosen from hydroxy(C₁-C₄)alkyl celluloses, preferably hydroxyethylcellulose and/or hydroxypropylcellulose.

10. Process according to claim 9, in which the non-associative non-ionic cellulose-based polymer(s) is (are) present in a total amount ranging preferably from 0.01 to 10% by weight, preferentially from 0.05 to 5% by weight and better still from 0.1 to 3% by weight relative to the total weight of composition A and/or composition B.

11. Process according to claim 1, in which the colouring agent(s) are present in a total amount ranging from 0.001 to 20%, preferably from 0.005 to 15% by weight, better still from 0.005 to 10% by weight relative to the total weight of composition A and/or composition B; preferably, the colouring agent(s) are chosen from pigments.

12. Process according to claim 1, in which the device for spraying composition A and/or composition B comprises at least one container containing composition A and/or composition B equipped with a spray mechanism provided with at least one dispensing orifice, in communication with the container.

13. Process according to claim 1, in which compositions A and/or B are packaged in one or more spray devices in the presence of at least one propellant.

14. Process according to claim 13, characterized in that said propellant is chosen from optionally halogenated volatile hydrocarbons, such as n-butane, propane, isobutane, pentane and halogenated derivatives thereof; carbon dioxide, nitrous oxide, dimethyl ether, nitrogen, compressed air, alone or as mixtures, preferably from alkanes, even more preferentially from n-butane, propane, isobutane and mixtures thereof.

15. Kit for dyeing keratin fibres such as the hair, comprising several compartments containing:
in a first compartment, a composition A comprising:
at least one alkoxysilane chosen from the compounds of formula (I) or of formula (I′) below, oligomers thereof and/or mixtures thereof as described according to claim 1, oligomers thereof and/or mixtures thereof; and
at least one alkoxysilane of formula (II), oligomers thereof and/or mixtures thereof;
in a second compartment, a composition B comprising at least one film-forming polymer,
composition A and/or composition B comprising at least one colouring agent chosen from pigments, direct dyes and mixtures thereof; and
composition A and/or composition B being packaged in one or more spray devices.
